(19) United States
(12) Patent Application Publication
(10) Pub. No.: US 2026/0008839 A1
(43) Pub. Date: Jan. 8, 2026

(54) Antibodies Blocking DLL4-Mediated Notch Signalling

(71) Applicant: The Brigham and Women's Hospital, Inc., Boston, MA (US)

(72) Inventors: Masanori Aikawa, Chestnut Hill, MA (US); Toshiaki Nakano, Fukuoka (JP); Shunsuke Katsuki, Brookline, MA (US)

(21) Appl. No.: 19/260,092

(22) Filed: Jul. 3, 2025

Related U.S. Application Data
(62) Division of application No. 17/294,147, filed on May 14, 2021, now Pat. No. 12,378,307.
(60) Provisional application No. 62/768,848, filed on Nov. 16, 2018.

(51) Int. Cl. C07K 16/18; G01N 33/563

(52) U.S. Cl. CPC C07K 16/18; G01N 33/563; C07K 2317/24; C07K 2317/565; C07K 2317/92

(57) ABSTRACT

The present invention is directed to antibodies that interact with DLL4 and inhibit it from binding to NOTCH receptors. The invention also includes nucleic acids encoding the antibodies and methods of using the antibodies in research and in the prevention or treatment of various diseases and conditions.

### CROSS REFERENCE TO RELATED APPLICATIONS

The present application is a divisional of U.S. patent application Ser. No. 17/294,147, filed on May 14, 2021, which is the U.S. National Phase Application under 35 U.S.C. § 371 of International Patent Application No. PCT/US2019/061534, filed on Nov. 14, 2019, which claims the benefit of U.S. Provisional Patent Application No. 62/768,848, filed on Nov. 16, 2018, which is hereby incorporated by reference in its entirety.

### STATEMENT OF GOVERNMENT FUNDING

This invention was made with government support under NIH Grant No. R01HL107550, awarded by the Department of Health and Human Services. The government has certain rights in the invention.

### SEQUENCE LISTING

This application contains a Sequence Listing that has been submitted electronically as an XML file named “29618-0340002_SL_ST26.XML.” The XML file, created on Jul. 3, 2025, is 60,924 bytes in size. The material in the XML file is hereby incorporated by reference in its entirety.

### FIELD OF THE INVENTION

The present invention is concerned with agents that can be used to modify the NOTCH signaling pathway of cells. In particular, it is concerned with antibodies that inhibit DLL4-induced NOTCH activity and the use of these antibodies in research, as well as in the prevention and treatment of a variety of diseases or conditions.

### BACKGROUND OF THE INVENTION

The NOTCH signaling pathway has been identified as playing an important role in many diverse biological functions, including differentiation and cellular proliferation (see U.S. Pat. No. 6,703,221). The pathway is activated by four different transmembrane receptor subtypes (designated as NOTCH-1-NOTCH-4) that have both an extracellular and intracellular domain (Nakano, et al., Atheroscl. Thromb. & Vascul. Biol. 36:2038-2047 (August 2016)). The extracellular portion of the receptors binds to ligands of the Jagged (Jagged1, Jagged2) and Delta-like (DLL1, DLL3, DLL4) families found on the surfaces of other cells. Following ligand binding, the receptor undergoes sequential cleavage by metalloproteases of the ADAM family (Bru, et al., Mol. Cell 5:207-216 (2000); Mumm, et al., Mol. Cell 5:197-206 (2000)) and the presenilin-dependent gamma-secretase (Selkoe, et al., Annu. Rev. Neurosci. 26:565-97 (2003); De Strooper, et al., Nature 398:518-522 (1999)). The final proteolytic cleavage step permits the intracellular domain of the NOTCH receptor to translocate to the cell nucleus where it interacts with transcription factors to induce target gene expression.

Over the last several years, evidence has accumulated that suggests that DLL4-mediated NOTCH signaling contributes to proinflammatory macrophage activation and to associated cardiovascular diseases, including atherosclerosis and vascular calcification (U.S. Pat. No. 8,133,857; Nakano, et al., Atheroscl. Thromb. & Vascul. Biol. 36:2038-2047 (August 2016), Nakano, et al., Circulation, in press). In addition, inhibition of DLL4-induced NOTCH activation has been associated with: vein graft disease (U.S. Pat. No. 9,567,396; Koga, et al. Atheroscl. Thromb. & Vascul. Biol. 35:2343-2353 (November 2015)); nonalcoholic fatty liver disease (US 2018/0230207); obesity (US 2016/0130334); metabolic disease (U.S. Pat. No. 8,889,131); type 1 and type 2 diabetes (Billiard, et al.; Cell Reports 22:895-904 (January 2018); cancer, including colorectal, breast and lung cancer (Gurney, et al., Vascular. Cell. 3:18 (2011); Kuhnert, et al., Vascular Cell. 3:20 (2011); Brzozowa, et al., Contemp. Oncol. 17:234-237 (2013); Kang, et al., Transl. Oncol. 11:599-608 (June 2018); Briot, et al.; Cancer Discov. 5:112-114 (February 2015)); tumor cell metastasis (Kuramoto, et al., Mol. Cancer Ther. 11(12):2578-2587 (December 2012)); and arthritis (Park, et al., Ann. Rheum. Dis. 2013;0:1-8. doi:10.1136/annrheumdis-2013-203467)).

Because of their substantial therapeutic potential, inhibitors of DLL4 signaling, and especially inhibitors that are relatively specific in their action, are of great interest.

### SUMMARY OF THE INVENTION

The present invention is based on the development of monoclonal antibodies that bind with high affinity to human DLL4 and that may be used to inhibit NOTCH receptor binding and NOTCH pathway activation. In some cases, the antibodies also have little or no effect on human DLL1. Thus, they may be used in research to help differentiate the effect of these NOTCH ligands and in patients without interfering with the normal biological action of DLL1. The antibodies also cross react with porcine DLL4.

The tables below are directed to six IgG type monoclonal antibodies that bind to DLL4 and that are structurally characterized with respect to light and heavy chain variable regions (and particularly complementarity determining regions (HCDRs and LCDRs) and framework regions (HFRs and LFRs) responsible for recognizing and interacting with this ligand. In some cases, information regarding an association rate constant (ka, in M⁻¹s⁻¹) and a dissociation rate constant (kd, in s⁻¹) for human DLL4 is provided either in the Table or a later description. These constants are for assays performed in 10 mM HEPES buffer (pH 7.4), 150 mM NaCl, 3 mM EDTA, 0.05% P20 (polyoxyethylene-sorbitan).

The present invention includes all antibodies with any combination of elements recited in a single table or in different tables. For example, the invention includes all combinations of light and heavy chain variable regions (and particularly complementarity determining regions (HCDRs and LCDRs) and framework regions (HFRs and LFRs) shown in the tables. Preferably the antibodies are IgG antibodies (especially monoclonal antibodies), that bind to human DLL4 with an affinity at least 1000 times (and preferably at least 10,000 or 100,000 times) higher than to human DLL1.

TABLE 1
Monoclonal Antibody 2H10
(little or no binding to human DLL1)

| | Nucleotides | Amino Acids |
|---|---|---|
| | Source: | Source: SEQ |
| Region | SEQ ID NO: 1 | ID NO: 2 |
| Heavy chain variable | 31-390 | 1-120 |
| region | | |
| HFR1-2H10 | 31-120 | 1-30 |
| HCDR1-2H10 | 121-135 | 31-35 |
| HFR2-2H10 | 136-177 | 36-49 |
| HCDR2-2H10 | 178-225 | 50-65 |
| HFR3-2H10 | 226-321 | 66-97 |
| HCDR3-2H10 | 322-363 | 98-111 |
| HFR4-2H10 | 364-390 | 112-120 |
| | Nucleotides | Amino Acids |
| | Source: | Source: SEQ |
| Region | SEQ ID NO: 3 | ID NO: 4 |
| Light chain variable | 3-311 | 1-103 |
| region | | |
| LFR1-2H10 | 3-53 | 1-17 |
| LCDR1-2H10 | 54-86 | 18-28 |
| LFR2-2H10 | 87-131 | 29-43 |
| LCDR2-2H10 | 132-152 | 44-50 |
| LFR3-2H10 | 153-248 | 51-83 |
| LCDR3-2H10 | 249-275 | 84-91 |
| LFR4-2H10 | 276-311 | 92-103 |

TABLE 2
Monoclonal Antibody 5D7

| | Nucleotides | Amino Acids |
|---|---|---|
| | Source: | Source: SEQ |
| Region | SEQ ID NO: 5 | ID NO: 6 |
| Heavy chain variable | 55-414 | 1-120 |
| region | | |
| HFR1-5D7 | 55-144 | 1-30 |
| HCDR1-5D7 | 145-159 | 31-35 |
| HFR2-5D7 | 160-201 | 36-49 |
| HCDR2-5D7 | 202-249 | 50-65 |
| HFR3-5D7 | 250-345 | 66-97 |
| HCDR3-5D7 | 346-381 | 98-109 |
| HFR4-5D7 | 382-414 | 110-120 |
| | Nucleotides | Amino Acids |
| | Source: | Source: SEQ |
| Region | SEQ ID NO: 7 | ID NO: 8 |
| Light chain variable | 23-352 | 1-110 |
| region | | |
| LFR1-5D7 | 23-88 | 1-22 |
| LCDR1-5D7 | 89-133 | 23-37 |
| LFR2-5D7 | 134-178 | 38-52 |
| LCDR2-5D7 | 179-199 | 53-59 |
| LFR3-5D7 | 200-295 | 60-91 |
| LCDR3-5D7 | 296-322 | 92-100 |
| LFR4-5D7 | 323-352 | 101-110 |

TABLE 3
Monoclonal Antibody 8B2

| | Nucleotides | Amino Acids |
|---|---|---|
| | Source: | Source: SEQ |
| Region | SEQ ID NO: 9 | ID NO: 10 |
| Heavy chain variable | 1-342 | 1-114 |
| region | | |
| HFR1-8B2 | 1-57 | 1-19 |
| HCDR1-8B2 | 58-87 | 20-29 |
| HFR2-8B2 | 88-129 | 30-43 |
| HCDR2-8B2 | 130-159 | 44-53 |
| HFR3-8B2 | 160-276 | 54-92 |
| HCDR3-8B2 | 277-309 | 93-103 |
| HFR4-8B2 | 310-342 | 104-114 |
| | Nucleotides | Amino Acids |
| | Source: | Source: SEQ |
| Region | SEQ ID NO: 11 | ID NO: 12 |
| Light chain variable | 141-461 | 1-107 |
| region | | |
| LFR1-8B2 | 141-209 | 1-23 |
| LCDR1-8B2 | 210-242 | 24-34 |
| LFR2-8B2 | 243-287 | 35-49 |
| LCDR2-8B2 | 288-308 | 50-56 |
| LFR3-8B2 | 309-404 | 57-88 |
| LCDR3-8B2 | 405-431 | 89-97 |
| LFR4-8B2 | 432-461 | 98-107 |

TABLE 4
Monoclonal Antibody 8C2
For human DLL4, ka greater than 5.0 × 10⁵;
kd less than 5.0 × 10⁻³
(little or no binding to human DLL1)

| | Nucleotides | Amino Acids |
|---|---|---|
| | Source: | Source: SEQ |
| Region | SEQ ID NO: 13 | ID NO: 14 |
| Heavy chain variable | 33-374 | 1-114 |
| region | | |
| HFR1-8C2 | 33-122 | 1-30 |
| HCDR1-8C2 | 123-137 | 31-35 |
| HFR2-8C2 | 138-179 | 36-49 |
| HCDR2-8C2 | 180-227 | 50-65 |
| HFR3-8C2 | 228-317 | 66-95 |
| HCDR3-8C2 | 318-341 | 96-103 |
| HFR4-8C2 | 342-374 | 104-114 |

| | Nucleotides | Amino Acids |
|---|---|---|
| | Source: | Source: SEQ |
| Region | SEQ ID NO: 15 | ID NO: 16 |
| Light chain variable | 1-318 | 1-106 |
| region | | |
| LFR1-8C2 | 1-69 | 1-23 |
| LCDR1-8C2 | 70-102 | 24-34 |
| LFR2-8C2 | 103-147 | 35-49 |
| LCDR2-8C2 | 148-168 | 50-56 |
| LFR3-8C2 | 169-264 | 57-88 |
| LCDR3-8C2 | 265-291 | 89-97 |
| LFR4-8C2 | 292-318 | 98-106 |

TABLE 5
Monoclonal Antibody 8D2
For human DLL4, ka greater than 1.0 × 10⁵;
kd less than 5.0 × 10⁻³
(little or no binding to human DLL1)

| | Nucleotides | Amino Acids |
|---|---|---|
| | Source: | Source: SEQ |
| Region | SEQ ID NO: 17 | ID NO: 18 |
| Heavy chain variable | 1-345 | 1-115 |
| region | | |
| HFR1-8D2 | 1-72 | 1-24 |
| HCDR1-8D2 | 73-87 | 25-29 |
| HFR2-8D2 | 88-129 | 30-43 |
| HCDR2-8D2 | 130-180 | 44-60 |
| HFR3-8D2 | 181-276 | 61-92 |
| HCDR3-8D2 | 277-312 | 93-104 |
| HFR4-8D2 | 313-345 | 105-110 |

| | Nucleotides | Amino Acids |
|---|---|---|
| | Source: | Source: SEQ |
| Region | SEQ ID NO: 19 | ID NO: 20 |
| Light chain variable | 36-365 | 1-110 |
| region | | |
| LFR1-8D2 | 36-101 | 1-22 |
| LCDR1-8D2 | 102-146 | 23-37 |
| LFR2-8D2 | 147-191 | 38-52 |
| LCDR2-8D2 | 192-212 | 53-59 |
| LFR3-8D2 | 213-308 | 60-91 |
| LCDR3-8D2 | 309-335 | 92-100 |
| LFR4-8D2 | 336-365 | 101-110 |

TABLE 6
Monoclonal Antibody 10C5

| | Nucleotides | Amino Acids |
|---|---|---|
| | Source: | Source: SEQ |
| Region | SEQ ID NO: 21 | ID NO: 22 |
| Light chain variable | 1-306 | 1-102 |
| region | | |
| LFR1-10C5 | 1-54 | 1-18 |
| LCDR1-10C5 | 55-87 | 19-29 |
| LFR2-10C5 | 88-132 | 30-44 |
| LCDR2-10C5 | 133-153 | 45-51 |
| LFR3-10C5 | 154-249 | 52-83 |
| LCDR3-10C5 | 250-276 | 84-92 |
| LFR4-10C5 | 277-306 | 93-102 |

The invention is directed, in part, to any IgG type antibodies or fragments thereof, especially monoclonal antibodies, with characteristics similar to those in the above tables. Specifically, the invention includes the following:

### A. Antibodies With Characteristics Based on Table 4

The invention includes any IgG antibodies, or antibody fragments, preferably monoclonal antibodies or fragments, that bind to human DLL4 with an affinity at least 1000 times (and preferably at least 10,000 or 100,000 times) higher than to human DLL1, and that comprise: a) a heavy chain variable region at least 90% (and preferably 95% or 98%) identical to the sequence of amino acids 1-114 of SEQ ID NO:14 and/or which includes one or more heavy chain complementarity determining regions (HCDRs) selected from the group consisting of: HCDR1-8C2, amino acids 31-35 of SEQ ID NO:14; HCDR2-8C2, amino acids 50-65 of SEQ ID NO:14; and HCDR3-8C2, amino acids 96-103 of SEQ ID NO:14; and b) a light chain variable region at least 90% (and preferably 95% or 98%) identical to the sequence of amino acids 1-106 of SEQ ID NO:16 and/or which includes one or more light chain complementarity determining regions (LCDRs) selected from the group consisting of: LCDR1-8C2, amino acids 23-34 of SEQ ID NO:16; LCDR2-8C2, amino acids 50-56 of SEQ ID NO:16; and LCDR3-8C2, amino acids 89-97 of SEQ ID NO:16.

In a preferred embodiment, antibodies or antibody fragments have a heavy chain variable region comprising HCDR1-8C2, HCDR2-8C2, and HCDR3-8C2 and a light chain variable region comprising LCDR1-8C2, LCDR2-8C2, and LCDR3-8C2. In addition, the heavy and light chain variable regions may include any or all of the framework sequences described in Table 4.

The invention also includes nucleic acids encoding the antibodies, or antibody fragments, that, preferably, have a heavy chain variable region encoded by nucleotides 33-374 of SEQ ID NO:13 and a light chain variable region encoded by nucleotides 1-318 of SEQ ID NO:15. Preferred nucleic acid sequences encoding heavy chain CDRs are: for HCDR1-8C2, nucleotides 123-137 of SEQ ID NO:13; for HCDR2-8C2 nucleotides 180-227 of SEQ ID NO:13; and for HCDR3-8C2 nucleotides 318-341 of SEQ ID NO:13. Preferred nucleic acid sequences encoding light chain CDRs are: for LCDR1-8C2, nucleotides 70-102 of SEQ ID NO:15; for LCDR2-8C2, nucleotides 148-168 of SEQ ID NO:15; and for LCDR3-8C2, nucleotides 265-291 of SEQ ID NO:15.

### B. Antibodies With Characteristics Based on Table 5

In a second aspect, the invention includes any IgG antibodies or antibody fragments, preferably monoclonal antibodies or fragments, that bind to human DLL4 with an affinity at least 1000 times (and preferably at least 10,000 or 100,000 times) higher than to human DLL1, and that comprise: a) a heavy chain variable region at least 90% (and preferably 95% or 98%) identical to the sequence of amino acids 1-115 of SEQ ID NO:18 and/or which includes one or more HCDRs selected from the group consisting of: HCDR1-8D2, amino acids 25-29 of SEQ ID NO:18; HCDR2-8D2, amino acids 44-60 of SEQ ID NO:18; and HCDR3-8D2, amino acids 93-104 of SEQ ID NO:18; and b) a light chain variable region at least 90% (and preferably 95% or 98%) identical to the sequence of amino acids 1-110 of SEQ ID NO:20 and/or which includes one or more LCDRs selected from the group consisting of: LCDR1-8D2, amino acids 23-37 of SEQ ID NO:20; LCDR2-8D2, amino acids 53-59 of SEQ ID NO:20; and LCDR3-8D2, amino acids 92-100 of SEQ ID NO:20.

In a preferred embodiment, antibodies, or antibody fragments, have a heavy chain variable region comprising: HCDR1-8D2; HCDR2-8D2; and HCDR3-8D2; and a light chain variable region comprising: LCDR1-8D2; LCDR2-8D2; and LCDR3-8D2. In addition, the heavy and light chain variable regions may include any or all of the framework sequences described in Table 5.

The invention also includes nucleic acids encoding the antibodies, or antibody fragments, that have a heavy chain variable region encoded by nucleotides 1-345 of SEQ ID NO:17 and a light chain variable region encoded by nucleotides 36-365 SEQ ID NO:19. Preferred nucleic acid sequences encoding heavy chain CDRs are: for HCDR1-8D2 nucleotides 73-87 of SEQ ID NO:17; for HCDR2-8D2, nucleotides 130-180 of SEQ ID NO:17; and for HCDR3-8D2, nucleotides 277-312 of SEQ ID NO:17. Preferred nucleic acid sequences encoding light chain CDRs are: for LCDR1-8D2, nucleotides 102-146 of SEQ ID NO:19; for LCDR2-8D2 nucleotides 192-212 of SEQ ID NO:19; and for LCDR3-8D2 nucleotides or 309-335 of SEQ ID NO:19.

### C. Antibodies With Characteristics Based on Table 1

In a third aspect, the invention includes any IgG antibodies or antibody fragments, preferably monoclonal antibodies or fragments, that bind to human DLL4 with an affinity at least 1000 times (and preferably at least 10,000 or 100,000 times) higher than human DLL1, and that comprise: a) a heavy chain variable region at least 90% (and preferably 95% or 98%) identical to the sequence of amino acids 1-120 of SEQ ID NO:2 and/or which includes one or more HCDRs selected from the group consisting of: HCDR1-2H10, amino acids 31-35 of SEQ ID NO:2; HCDR2-2H10, amino acids 50-65 of SEQ ID NO:2; and HCDR3-2H10,amino acids 98-111 of SEQ ID NO:2; and a light chain variable region at least 90% identical to the sequence of amino acids 1-103 of SEQ ID NO:4 and/or which includes one or more LCDRs selected from the group consisting of LCDR1-2H10, amino acids 18-28 of SEQ ID NO:4; LCDR2-2H10, amino acids 44-50 of SEQ ID NO:4; and LCDR3-2H10, amino acids 84-91 of SEQ ID NO:4.

In a preferred embodiment, antibodies or antibody fragments, have a heavy chain variable region comprising: HCDR1-2H10; HCDR2-2H10; and HCDR3-2H10; and a light chain variable region comprising: LCDR1-2H10; LCDR2-2H10; and LCDR3-2H10. In addition, the heavy and light chain variable regions may include any or all of the framework sequences described in Table 1.

The invention also includes nucleic acids encoding the antibodies, or antibody fragments, that have a heavy chain variable region preferably encoded by nucleotides 31-390 of SEQ ID NO:1 and a light chain variable region preferably encoded by nucleotides 3-311 of SEQ ID NO:3. Preferred nucleic acid sequences encoding heavy chain CDRs are: for HCDR1-2H10, nucleotides 121-135 of SEQ ID NO:1; for HCDR2-2H10, nucleotides 178-225 of SEQ ID NO:1; and for HCDR3-2H10, nucleotides 322-363 of SEQ ID NO:1. Preferred nucleic acid sequences encoding light chain CDRs are: for LCDR1-2H10, nucleotides 54-86 of SEQ ID NO:3; for LCDR2-2H10, nucleotides 132-152 of SEQ ID NO:3; and for LCDR3-2H10, nucleotides 249-275 of SEQ ID NO:3.

### D. Antibodies With Characteristics Based on Table 2

In a fourth aspect, the invention includes any IgG antibodies or antibody fragments, preferably monoclonal antibodies or fragments, that bind to human DLL4 and which comprise: a) a heavy chain variable region at least 90% identical to the sequence of amino acids 1-120 of SEQ ID NO:6 and/or which includes one or more HCDRs selected from the group consisting of HCDR1-5D7, amino acids 31-35 of SEQ ID NO:6; HCDR2-5D7, amino acids 50-65 of SEQ ID NO:6; and HCDR3-5D7, amino acids 98-109 of SEQ ID NO:6; and b) a light chain variable region at least 90% identical to the sequence of amino acids 1-110 of SEQ ID NO:8 and/or which includes one or more LCDRs selected from the group consisting of: LCDR1-5D7, amino acids 23-37 of SEQ ID NO:8; LCDR2-5D7, amino acids 53-59 of SEQ ID NO:8; and LCDR3-5D7, amino acids 92-100 of SEQ ID NO:8.

In a preferred embodiment, antibodies or antibody fragments have a heavy chain variable region comprising: HCDR1-5D7; HCDR2-5D7; and HCDR3-5D7; and a light chain variable region comprising: LCDR1-5D7; LCDR2-5D7; and LCDR3-5D7. In addition, the heavy and light chain variable regions may include any or all of the framework sequences described in Table 2.

The invention also includes nucleic acids encoding the antibodies, or antibody fragments, that have a heavy chain variable region encoded by nucleotides 55-414 of SEQ ID NO:5 and a light chain variable region encoded by nucleotides 23-352 SEQ ID NO:7. Preferred nucleic acid sequences encoding heavy chain CDRs are: for HCDR1-5D7, nucleotides 145-159 of SEQ ID NO:5; for HCDR2-5D7, nucleotides 202-249 of SEQ ID NO:5; and for HCDR3-5D7, nucleotides 346-381 of SEQ ID NO:5. Preferred nucleic acid sequences encoding light chain CDRs are: for LCDR1-5D7, nucleotides 89-133 of SEQ ID NO:7; for LCDR2-5D7, nucleotides 179-199 of SEQ ID NO:7; and for LCDR3-5D7, nucleotides 296-322 of SEQ ID NO:7.

### E. Antibodies With Characteristics Based on Table 3

In a fifth aspect, the invention includes any IgG antibodies or antibody fragments, preferably monoclonal antibodies or fragments, that bind to human DLL4 and that comprise: a) a heavy chain variable region at least 90% identical to the sequence of amino acids 1-114 of SEQ ID NO:10 and/or which includes one or more HCDRs selected from the group consisting of HCDR1-8B2, amino acids 20-29 of SEQ ID NO: 10; HCDR2-8B2, amino acids 44-53 of SEQ ID NO: 10; and HCDR3-8B2, amino acids 93-103 of SEQ ID NO:10; and b) a light chain variable region at least 90% identical to the sequence of amino acids 1-107 of SEQ ID NO:12 and/or which includes one or more LCDRs selected from the group consisting of LCDR1-8B2, amino acids 24-34 of SEQ ID NO:12; LCDR2-8B2, amino acids 50-56 of SEQ ID NO:12; and LCDR3-8B2, amino acids 89-97 of SEQ ID NO:12.

In a preferred embodiment, antibodies or antibody fragments, have a heavy chain variable region that comprises: HCDR1-8B2; HCDR2-8B2; and HCDR3-8B2; and the light chain variable region that comprises: LCDR1-8B2; LCDR2-8B2; and LCDR3-8B2. In addition, the heavy and light chain variable regions may include any or all of the framework sequences described in Table 3.

The invention also includes nucleic acids encoding the antibodies, or antibody fragments, that have a heavy chain variable region encoded by nucleotides 1-342 of SEQ ID NO:9 and a light chain variable region encoded by nucleotides 141-461 SEQ ID NO:11. Preferred nucleic acid sequences encoding heavy chain CDRs are: for HCDR1-8B2,nucleotides 58-87 of SEQ ID NO:9; for HCDR2-8B2, nucleotides 130-159 of SEQ ID NO:9; and for HCDR3-8B2, nucleotides 277-309 of SEQ ID NO:9. Preferred nucleic acid sequences encoding light chain CDRs are: for LCDR1-8B2, nucleotides 210-242 of SEQ ID NO:11; for LCDR2-8B2, nucleotides 288-308 of SEQ ID NO:11 and for LCDR3-8B2, nucleotides or 405-431 of SEQ ID NO:11.

### F. Antibodies With Characteristics Based on Table 6

In a sixth aspect, the invention includes any IgG antibodies or antibody fragments, preferably monoclonal antibodies or fragments, that bind to human DLL4 and that comprise: a light chain variable region at least 90% identical to the sequence of amino acids 1-102 of SEQ ID NO:22 and/or which includes one or more LCDRs selected from the group consisting of LCDR1-10C5, amino acids 19-29 of SEQ ID NO:22; LCDR2-10C5, amino acids 45-51 of SEQ ID NO:22; and LCDR3-10C5, amino acids 84-92 of SEQ ID NO:22.

In a preferred embodiment, antibodies or antibody fragments have a light chain variable region that comprises: LCDR1-10C5, LCDR2-10C5, and LCDR3-10C5. In addition, the light chain variable region may include any or all of the framework sequences described in Table 6.

The invention also includes nucleic acids encoding the antibodies or antibody fragments that have a light chain variable region encoded by nucleotides 1-306 SEQ ID NO:21. Preferred nucleic acid sequences encoding light chain CDRs are: for LCDR1-10C5 nucleotides 55-87 of SEQ ID NO:21; for LCDR2-10C5, nucleotides 133-153 of SEQ ID NO:21; and for LCDR3-10C5, nucleotides 250-276 of SEQ ID NO:21.

### G. Common Characteristics and Therapeutic Uses of Antibodies

All of the antibodies or antibody fragments described above (preferably monoclonal antibodies or fragments) should have an association rate constant, ka (1/Ms), for human DLL4 of 1.0×10⁴ or higher (preferably 5.0×10⁴ or higher and more preferably 1×10⁵ or higher) and a dissociation rate constant, kd (1/s) of 5×10⁻³ or less (and preferably 1.0×10⁻³ or less).

Monoclonal antibodies may be nonhuman (e.g., mouse or rat antibodies), chimeric, humanized or fully human. Any of these different types of antibodies may be used for research purposes but humanized monoclonal antibodies (in which mouse CDR and, optionally also FR, regions replace the corresponding sites in human antibodies) or fully human monoclonal antibodies are preferred for use therapeutically in people, with fully human monoclonals being most preferred. Similarly, humanized or fully human antibody fragments (as opposed, for example, to nonhuman antibody fragments) are preferred for the treatment of human patients.

The antibodies or fragments of the antibodies may be modified to include any of the post-translational modifications that are known in the art and commonly applied to antibodies, provided that the modified antibodies or fragments maintain specificity for binding to human or porcine DLL4. Modifications may include PEGylation, phosphorylation, methylation, acetylation, ubiquitination, nitrosylation, glycosylation, ADP-ribosylation, or lipidation. Alternatively, or in addition, the antibodies or fragments may further comprise a detectable label that can be used to detect binding in an immunoassay. Labels that may be used include radioactive labels, fluorophores, chemiluminescent labels, enzymatic labels (e.g., alkaline phosphatase or horseradish peroxidase); biotin; avidin; and heavy metals.

The antibodies or fragments may be used in the treatment or prevention of diseases or conditions in humans that are caused or exacerbated by the binding of DLL4 to NOTCH receptors. Typically, this will involve administering a therapeutically effective amount of the antibody systemically or locally to a patient. The antibody is preferably administered by injection as part of a pharmaceutical composition that also includes a pharmaceutically acceptable carrier. Other active agents or excipients may also be included in the compositions administered. Diseases or conditions that may be treated or prevented include: cardiovascular disease, including atherogenesis, atherosclerosis and vascular calcification; vein graft disease; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

### H. Use in Assays

The antibodies or antibody fragments described herein may be used to bind to DLL4 (preferably human or porcine DLL4) as part of any of the immunoassays that are commonly used in the art. In some assays (typically called “one step” or “direct” assays) the antibodies or fragments will include a detectable label such as a radioactive label, fluorophore, chemiluminescent label, or enzymatic label. In other assays (typically called “two step” or “indirect” assays), the unlabeled antibody or fragment will constitute a “first” antibody and will bind to DLL4. Then a “second” antibody that has a detectable label will be used to bind to the first antibody. In a variation of a two step assay, the first antibody or fragment will include a binding agent such as biotin and then bind to a complementary binding agent, e.g. avidin, that is, or has been modified to be, detectable. Many variations on these assays have been described in the art and can be used. Examples of specific types of immunoassays include immunohistochemistry, Western blots, fluorescence-activated cell sorting, chemiluminescence immunoassays, radioimmunoassays, enzyme linked immunosorbent assays, immunoprecipitation assays, and immunoelectron microscopy.

In a preferred assay, DLL4-expressing cells in a test sample from a subject may be detected or quantitated by assaying the sample for cells expressing DLL4 using one of the assays discussed above. The test sample may be a bodily fluid (e.g., blood, plasma or serum) or it may be a tissue or cell sample obtained, for example, by biopsy or autopsy. The subject may be a laboratory animal or a human and may either be normal (i.e., disease free) or have a disease or condition selected from the group consisting of: cardiovascular disease; vein graft disease; arteriovenous fistula failure; nonalcoholic fatty liver disease; obesity; metabolic disease; type 1 or type 2 diabetes; cancer; and transplanted tissues or organs.

### I. Use as Research Tools

Apart from being used therapeutically and in the assays discussed above, the antibodies or antibody fragments described herein may be used by researchers studying the effects of DLL-4. For example, antibodies might be administered to test animals during development to help determine the effect that DLL4-mediated NOTCH pathway activation has on this process. Studies might be also be used to examine DLL4′s contribution to disease processes or to look at effects on the activities of specific types of cells.

### BRIEF DESCRIPTION OF THE DRAWINGS

FIG. 1A and 1B: FIGS. 1A and 1B show the results from experiments in which DLL4-expressing Cf2Th cells were combined with control IgG or serum, followed by recombinant NOTCH1-Fc and PE anti-IgG Fc. In FIG. 1B, results are merged from three independent determinations.

FIG. 2: FIG. 2 shows the results of three experiments in which DLL4 binding assays (using supernatants) were performed.

FIG. 3A and 3B: FIGS. 3A and 3B show the result of DLL4 binding assays performed using purified antibodies and Cf2Th cells. The DLL4-expressing Cf2Th cells were added with neutralizing DLL4 antibodies (or control IgG), followed by recombinant NOTCH1-Fc and PE anti-IgG Fc. The results shown in FIG. 3B are based on three independent experiments.

FIG. 4A and 4B: FIG. 4A and 4B show the results of a DLL4 binding assay performed using purified antibodies and HEK293T cells. The DLL4-expressing HEK293T cells were added with neutralizing DLL4 antibodies (or control IgG), followed by recombinant NOTCH1-Fc and PE anti-IgG Fc. The results shown in FIG. 4B are based on three independent experiments.

FIG. 5: FIG. 5 shows the results of an RBP-Jκ reporter assay. RBP-Jκ is a key transcription factor of the pan NOTCH pathway. NOTCH signaling activates RBP-Jκ. Recombinant human DLL4 was coated on the culture plates (“immobilized DLL4”). Neutralizing DLL4 antibodies or control IgG were added in the medium. The RBP-Jκ reporter construct was transfected in the mouse macrophage cell line RAW264.7. RAW264.7 cells were then seeded on culture plates coated with recombinant DLL4. After 48 hours. The relative luciferase values (RPB-Jκ firefly luciferase/control CMV renilla luciferase) were measured. The results shown are based on three independent experiments.

FIG. 6: FIG. 6 was part of a study examining whether the monoclonal antibodies 8D2 and 8C2 recognize the same, or overlapping, epitopes on either human or porcine DLL4. In the figure shown, the surface of a Biacore chip was coated with human DLL4 and the monoclonal antibody 8D2 was then allowed to bind to the immobilized DLL4 until all binding sites appeared to be saturated. The chip was then exposed to the monoclonal antibody 8C2while measuring changes in surface plasmon resonance.

FIG. 7: FIG. 7 shows the sensor readings obtained from an experiment similar to that described in FIG. 6 except that porcine DLL4 was used to coat the surface of the Biacore chip instead of human DLL4.

FIG. 8: FIG. 8 shows the results of an experiment that is essentially the same as that described in FIG. 6, except that the order of addition of 8D2 and 8C2 was reversed, i.e., 8C2 was bound to immobilized human DLL4 and 8D2 was then introduced.

FIG. 9: FIG. 9 shows the results of an experiment that is essentially the same as that described in FIG. 7, except that the order of addition of 8D2 and 8C2 was reversed, i.e., 8C2 was bound to immobilized porcine DLL4 and 8D2 was then introduced.

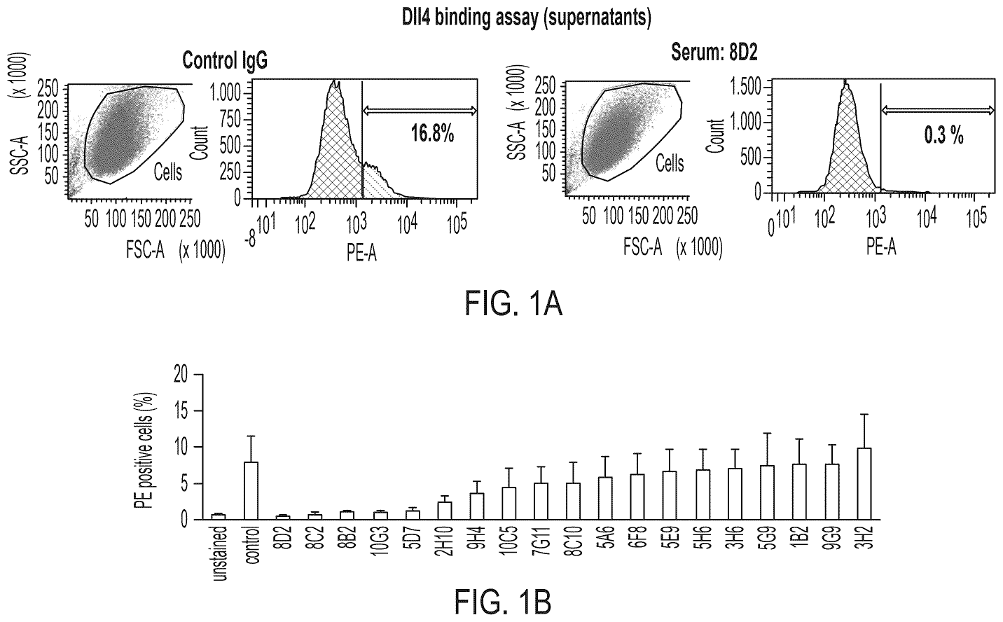

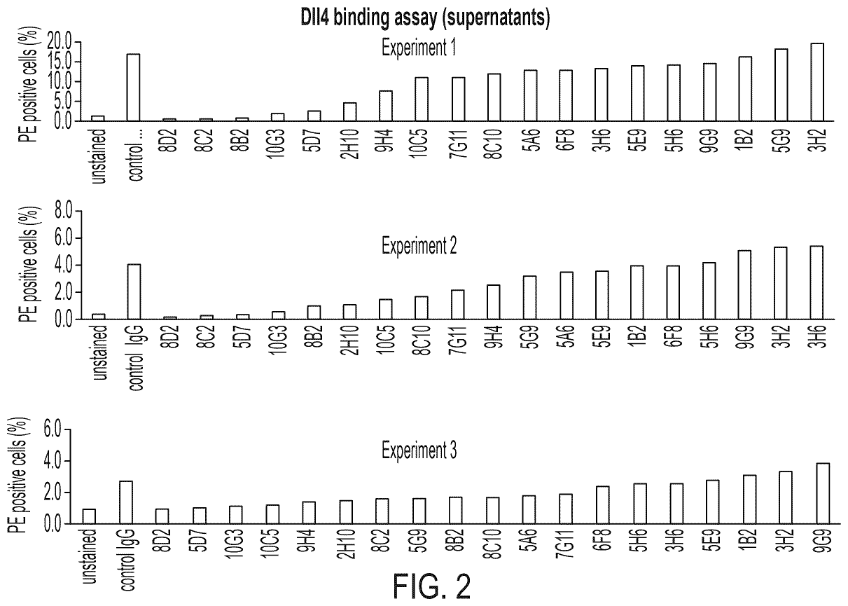

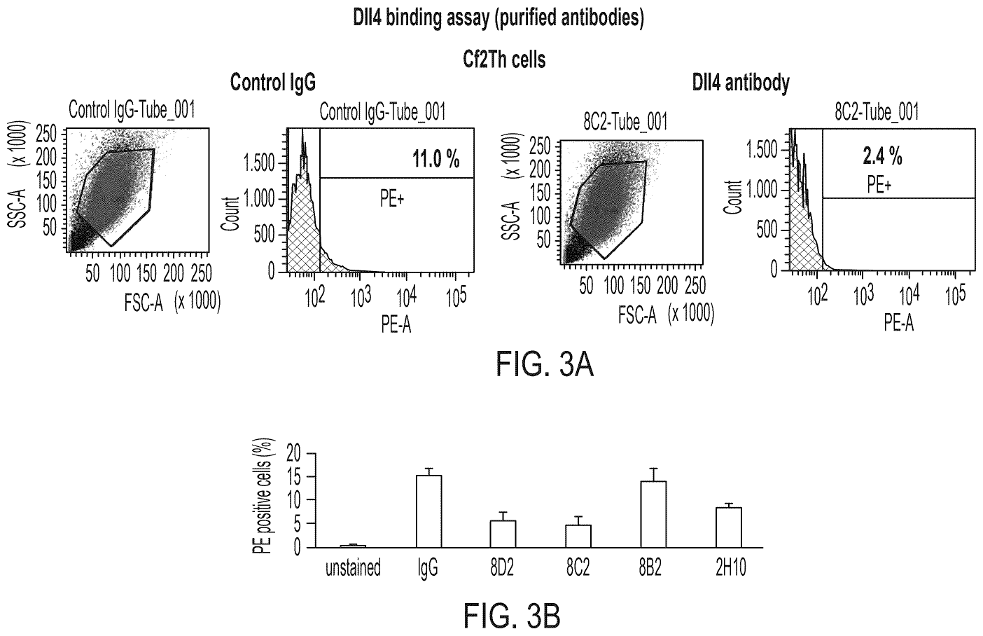

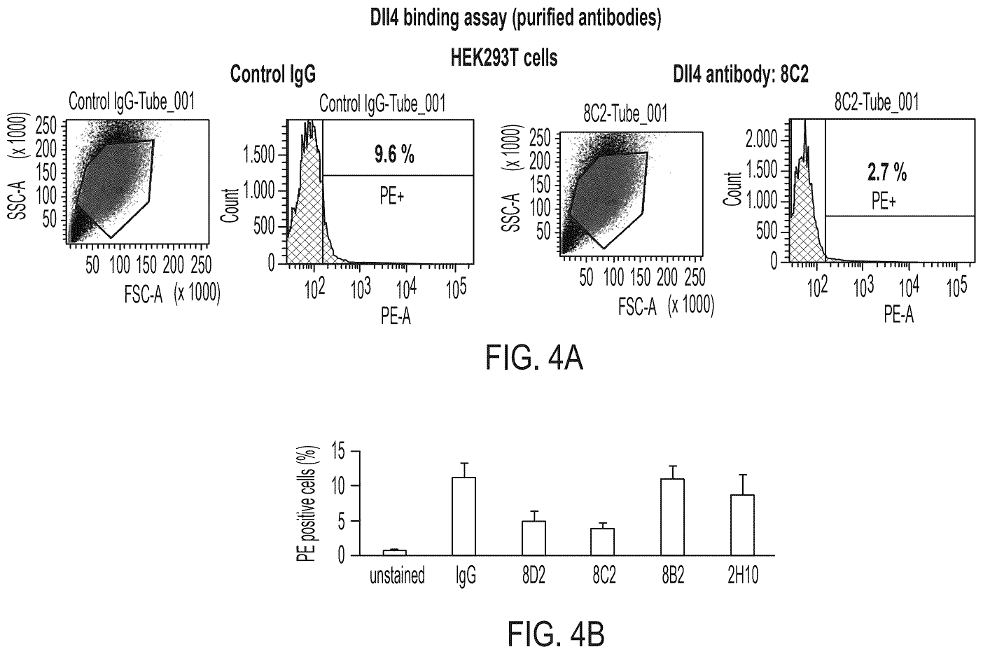

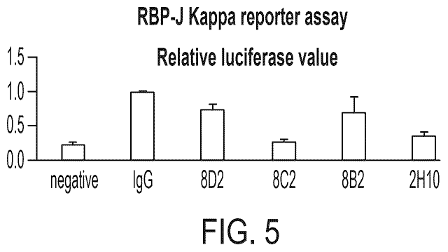

### DETAILED DESCRIPTION OF THE INVENTION

### A. Definitions

Antibody: The term “antibody” as used herein refers to whole antibodies (preferably of the IgG family) that bind to human and or porcine DLL4 and that, when bound, inhibit the binding of DLL4 to NOTCH receptors and the associated NOTCH signaling. Structurally, the intact antibody has two heavy chains and two light chains that are joined together by disulfide bonds. The heavy chains typically each have a constant region (which is divided into CH1, CH2 and CH3 domains) and a variable region which has three hypervariable regions, also called complementarity determining regions (CDRs), that interact directly with DLL4, and that are interspersed with four framework regions (FRs) that are somewhat more conserved. The CDRs and FRs abut one another and are arranged from amino-terminus to carboxy-terminus as: FR1, CDR1, FR2, CDR2, FR3, CDR3, FR4. The light chains have a constant region and a variable region that, like the heavy chain variable region, has three CDRs that interact directly with DLL4 and that are interspersed in the same manner with four framework regions. It is the variable regions of the heavy and light chains that together form a binding site for DLL4. Antibodies or antibody fragments may undergo post translational modifications that are well known in the art including: PEGylation, phosphorylation, methylation, acetylation, ubiquitination, nitrosylation, glycosylation, and lipidation. It will be understood that the invention includes all such routine modifications so long as the antibodies or fragments maintain the ability to bind with specificity to DLL4.

Antibody Fragment: The phrase “antibody fragment,” as used herein, refers to one or more portions of an antibody that retain the ability to specifically interact with DLL4 and inhibit it from binding to NOTCH receptors. Examples of binding fragments include, but are not limited to, Fab fragments, Fab₂ fragments, and fragments comprising two Fab fragments linked by a disulfide bond.

Isolated antibody: The phrase “isolated antibody,” “isolated monoclonal antibody,” “isolated antibody fragment” etc. refers to antibodies, fragments etc. that are substantially (at least 90%) free of other antibodies or fragments having different antigenic specificities and substantially free of cellular material.

“Specific binding” or “specificity”: As used herein, reference to an antibody that exhibits specific binding or that binds with specificity means that the antibody binds to DLL4 with an equilibrium constant (K_{A}) (kₒₙ/k_{off}) of at least of at least 1.0×10⁴ M⁻¹; and preferably at least 1.0×10⁵ M⁻¹; 1.0×10⁶ M⁻¹; 1.0×10⁷ M⁻¹; 1.0×10⁸ M⁻¹; 1.0×10⁹ M⁻¹; or 1.0×10¹⁰ M⁻¹. Standard assays well known in the art (e.g., ELISAs, Western blots and RIAs) can be used to evaluate the binding of antibodies to DLL4 and the kinetics of binding assessed by, e.g., Biacore analysis, or FACS relative affinity (Scatchard).

Affinity: The term “affinity” refers to the strength of interaction between antibody and antigen. Within each antigenic site, the variable region of the antibody interacts through non-covalent bonds with antigen at numerous sites; the more interactions, the stronger the affinity.

Percent identical: The term percent identical when referring to two or more amino acid or nucleotide sequences refers to, depending on context, the percentage of nucleotides or amino acids that are the same over an entire sequence or a portion of a sequence when the sequences are aligned for maximum correspondence.

Therapeutically effective amount: The term “therapeutically effective amount” refers to a sufficient amount of a DLL4 antibody to prevent the onset, or retard the progression of a disease or condition, symptoms associated with a disease or condition or otherwise result in an improvement in an accepted characteristic of a disease or condition when administered according to a given treatment protocol.

### B. The Making of Antibodies

Methods for making the antibodies disclosed herein are described in the Examples section. However, other ways of making antibodies with similar characteristics can be used as well. For example, phage display is a method for making antibodies without using animals.

Also, based on the sequence information disclosed herein, VH/VL encoding nucleotide sequences can be synthesized and cloned into commercial vectors with a mouse/rat/human framework. Recombinant antibodies can then be produced either from a stable cell line or after transient transfection.

### C. Drug Formulation

The antibodies or antibody fragments described herein will typically be administered to patients in a pharmaceutical composition comprising the antibody or fragment along with a pharmaceutically acceptable carrier. The carrier may be any solvent, diluent, liquid or solid vehicle that is pharmaceutically acceptable and typically used in formulating drugs. Guidance concerning the making of pharmaceutical formulations can be obtained from standard works in the art (see, e.g., Remington's Pharmaceutical Sciences, 16th edition, E. W. Martin, Easton, Pa. (1980)). In addition, pharmaceutical compositions may contain any of the excipients that are commonly used in the art. Examples of carriers or excipients that may be present include, but are not limited to, sugars (e.g., lactose, glucose and sucrose); starches, such as corn starch or potato starch; cellulose and its derivatives (e.g., sodium carboxymethyl cellulose, ethyl cellulose, or cellulose acetate); malt; gelatin; talc; cocoa butter; oils (e.g., peanut oil, cottonseed oil, safflower oil, sesame oil, olive oil, corn oil, or soybean oil); glycols; buffering agents; saline; Ringer's solution; alcohols; lubricants; coloring agents; dispersing agents; coating agents; flavoring agents; preservatives; or antioxidants. Antibody or an antibody fragment may be the sole active ingredient in a composition or other therapeutically active agents may also be present.

The invention is compatible with the delivery of compounds by any route known in the art that permits antibody to remain intact and capable of binding with specificity to DLL4. Options include peroral, internal, rectal, nasal, lingual, transdermal, intravenous, intra-vascular, peri-vascular, intramuscular, intraperitoneal, intracutaneous and subcutaneous routes. The most preferred route is by injection. Injectable preparations may be in the form of sterile, injectable aqueous or oleaginous suspensions, diluents or solvents that may be used may include 1,3-butanediol, water, Ringer's solution and isotonic saline solutions. In addition, oils or fatty acids may be present. Grafts such as autologous vein grafts and tissue-engineered grafts may be treated with the antibody before implantation. Similarly, organs for transplantation may be treated with the antibody before surgery.

Liquid dosage forms for oral administration may include, but are not limited to, pharmaceutically acceptable emulsions, microemulsions, solutions, suspensions, syrups and elixirs. In addition to the active compounds, liquid dosage form may contain inert diluents commonly used in the art, such as, for example, water, or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, dimethyl formamide, oils, glycerol, alcohols, polyethylene glycols, and fatty acid esters.

### D. Dosage

Pharmaceutical compositions will typically be given to a patient in one or more unit dosage forms. A “unit dosage form” refers to a single drug administration entity, e.g., a single tablet, capsule or injection volume. The amount of antibody or antibody fragment present should be at least the amount required to reduce the binding of DLL4 at a selected site, for example by at least 10%, or at least 20%, 30%, 40%, 50%, 60% or 70%. On a biological level, sufficient antibody or antibody fragment should be administered in a dosage regimen and over a selected period to show an improvement in one or more clinically accepted measures of a disease or condition or to reduce symptoms associated with the disease or condition. For example, a sufficient amount of an antibody may be administered reduce vein graft rejection 20%, 40%, 60% or more during the first year after the graft is made. The exact dosages given and amount of antibody or antibody fragment in unit dosage forms may be determined for individual patients using methods that are well known in the art of pharmacology and may be further adjusted by physicians based on clinical considerations.

### E. Treatment Methods

The antibodies and antibody fragments described herein may be used in treating or preventing any disease or condition associated with the binding of DLL4 to NOTCH receptors. This includes, but is not limited to: cardiovascular disease, including atherogenesis, atherosclerosis and vascular calcification; vein graft disease; arteriovenous fistula failure, nonalcoholic fatty liver disease; obesity; metabolic disease; type 1 and type 2 diabetes; cancer, including colorectal, breast and lung cancer; tumor cell metastasis; and arthritis. The antibodies and antibody fragments described herein may be used in treating or preventing a failure or dysfunction of artificial or tissue-engineered tissues or organs such as artificial vascular grafts and tissue-engineered heart valves; and in preventing rejection or graft-versus-host disease following solid organ and cell transplantation. Subjects, particularly individuals having, or at high risk of developing, one or more such diseases or conditions (e.g., dyslipidemia, diabetes, chronic kidney disease, hypertension) may be treated by administering one or more of the antibodies or antibody fragments described herein. Antibodies or antibody fragments may also be given to test animals, particularly pigs, to study their effect on a disease or condition or the development of a disease or condition. Antibodies or fragments may be administered as the sole active agents in a dosage form, or they may be combined with other drugs to improve overall effectiveness.

### F. Use in Immunoassays

As discussed above, the antibodies and antibody fragments of the present invention may be used in the treatment or prevention of a variety of diseases and conditions. In addition, the invention includes immunoassays which use the antibodies or fragments. Procedures that may be used include immunohistochemistry, Western blots (also called immunoblots), procedures involving flow cytometry (e.g., fluorescence-activated cell sorting, “FACS”), chemiluminescence immunoassays, radioimmunoassays (RIAs), enzyme linked immunosorbent assays (ELISAs), “sandwich” immunoassays, immunoprecipitation assays, and immuno-electron microscopy. The antibodies or fragments may be joined to any type of detectable label commonly used in the art, including radioactive labels, fluorophores, chemiluminescent labels, enzymatic labels (e.g., alkaline phosphatase or horseradish peroxidase) and heavy metals.

The assays may be one step (direct) assays, in which the antibodies or fragments are detectably labeled or two step (indirect) assays in which the antibodies or fragments are not themselves labeled but are detected using a second antibody (or other specific binder) that does have a detectable label. There are many variations of these assays that have been described in the art that should be compatible with the present antibodies (sec e.g., Wild, David (ed.) (2013), The Immunoassay Handbook, 4th edition, Elsevier Science, Hardcover ISBN 9780080970370; Gosling, (2000) Immunoassays: A Practical Approach (Practical Approach Series) Oxford Univ Press; Diamandis; Evin, J. Pept. Sci. 1(2): 132-139 (1995)); Sernee, et al., Eur. J. Biochem. 270:495-506 (2003); and Pinnix, et al., J. Biol. Chem. 276:481-487 (2001); each of which is incorporated by reference herein in their entirety).

The assays may be used diagnostically or prognostically to detect and/or quantitate DLL4-expressing cells or a free form of DLL4 in biological samples, including biopsy samples, tissue samples, or samples of bodily fluid such as blood, serum or plasma. Among the diseases or conditions that assays may be used in connection with are: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; failure or dysfunction of artificial or tissue-engineered tissues or organs; and rejection or graft-versus-host disease after organ or cell transplantation. The assays may also be used in research to study DLL4-induced NOTCH activity in cells and test animals.

### EXAMPLES

### The Making of Antibodies

cDNAs encoding amino acid 27-529 (ECD) and separately of amino acid 151-221 (DSL domain) of human DLL4 were cloned into separate expression plasmids (Aldevron Freiburg GmbH, Freiburg, Germany). Groups of laboratory rats (Wistar) were immunized with both plasmids by intradermal application of DNA-coated gold-particles using a hand-held device for particle-bombardment (“gene gun”). Cell surface expression on transiently transfected HEK cells was confirmed with anti-tag antibodies recognizing a tag added to the N-terminus of the DLL4 protein. Serum samples were collected after a series of immunizations and tested by flow cytometry on HEK cells transiently transfected with the aforementioned expression plasmids. Antibody-producing cells were isolated and fused with mouse myeloma cells (Ag8) according to standard procedures. Hybridomas producing antibodies specific for DLL4 were identified by screening using human DLL4 ECD, then using pig DLL4 ECD and finally using human DLL1 (negative screening). Cell pellets of positive hybridomas cells were prepared using an RNA protection agent (RNAlater, cat. #AM7020 by ThermoFisher Scientific) and further processed for sequencing of the variable domains of the antibodies.

### Example 1: Antibody Binding to DLL4

These experiments examine the binding of monoclonal antibodies 8D2, 8C2 and 2H10 to human DLL4, human DLL1 and porcine DLL4

### A. Materials and Methods

### Materials

Equipment: Biacore 3000
Assay Buffer—10 mM HEPES buffer (pH 7.4), 150 mM NaCl, 3 mM EDTA, 0.05% P20 (polyoxyethylenesorbitan)
Regeneration Buffer—10 mM Glycine buffer (pH 1.75)
Conjugation Buffer—10 mM sodium acetate buffer (pH 5)
Flow rate—The flow rate used for capturing the ligand is 5 μl/min. The flow rate for kinetics analysis is 30 μl/min.

### Abbreviations

| | |
|---|---|
| CM5 chip | Carboxymethylated dextran coated chip |
| SPR | Surface Plasmon Resonance |
| RU | Response Units |
| KD | Equilibrium binding affinity constant. |
| | KD is defined as the analyte concentration |
| | at which 50% of the maximum available |
| | ligand is in complex form at equilibrium |
| Analyte | One of the interacting molecules flown |
| | over the surface |
| Ligand | One of the interacting molecules immobilized or |
| | captured on the surface |
| Rmax | Maximum binding capacity (in RU) of ligand |
| | captured/immobilized on the surface |
| MW | Molecular weight |
| HBS-EP | HEPES buffered saline supplemented with |
| | EDTA and P20 |
| ka | Association rate constant in M⁻¹s⁻¹ |
| kd | Dissociation rate constant in s⁻¹ |
| χ² | Chi squared |
| EDC | N-ethyl-N′-(3-dimethyl aminopropyl |
| | carbodiimide) |
| NHS | N-hydroxy succinamide |

### Procedures

Binding experiments were performed on Biacore 3000 at 25° C. Flow cell 2, 3 and 4 of the CM5 chip were coated with the goat anti-rat Fc Capture Ab using EDC/NHS the amine coupling method as per GE manufacturer's instruction. The unoccupied sites were blocked with 1M ethanolamine. Three test antibodies were captured at an RU as indicated on flow cell 2, 3 and 4. Ag was flowed over the chip. Binding of antigen to the antibodies was monitored in real time. From the observed kₒₙ and k_{off}, KD was determined. For the interactions with fast off rate, steady state kinetics was used to determine KD.

Scouting analysis was performed using a single analyte concentration of 100 nM. At this concentration, binding should be observed even if the ligand binding affinity is weak. Flow cell 1 response was used for reference subtraction.

Full kinetics was performed for control antibody with the range of analyte with a 2 fold serial dilution and flowed over the ligand from lowest to highest concentration range as indicated.

Chi square (X²) analysis was carried out between the actual sensorogram (colored line) and the sensorogram generated from the Analysis software (black line) to determine the accuracy of the analysis. X² within 1-2 is considered significant (accurate) and below 1 is highly significant (highly accurate).

### B. Results Phase I

### Scouting

TABLE E1

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-8D2-D10 | Hu.DLL1 | NA | NA | NA | 100 nM | NA | NA |
| (220 RU) | | | | | | | |

TABLE E2

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-8D2-D10 | Hu.DLL4 | 4.74 × 10⁵ | 1.08 × 10⁻³ | 117 | 100 nM | 2.27 × 10⁻⁹ | 6.91 |
| (220 RU) | | | | | | | |

TABLE E3

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-8C2-D10 | Hu.DLL1 | NA | NA | NA | 100 nM | NA | NA |
| (170 RU) | | | | | | | |

TABLE E4

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-8C2-D10 | Hu.DLL4 | 1.37 × 10⁵ | 5.05 × 10⁻⁴ | 101 | 100 nM | 3.68 × 10⁻⁹ | 0.0375 |
| (170 RU) | | | | | | | |

TABLE E5

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-2H10-A7 | Hu.DLL1 | NA | NA | NA | 100 nM | NA | NA |
| (161 RU) | | | | | | | |

TABLE E6

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-2H10-A7 | Hu.DLL4 | 1.24 × 10⁴ | 1.09 × 10⁻³ | 30.8 | 100 nM | 8.86 × 10⁻⁸ | 0.0487 |
| (161 RU) | | | | | | | |

Scouting repeat for DLL1 at higher ligand RU and analyte concentration:

TABLE E7

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-8D2-D10 | Hu.DLL1 | NA | NA | NA | 1000 nM | NA | NA |
| (556 RU) | | | | | | | |

TABLE E8

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-8C2-D10 | Hu.DLL1 | NA | NA | NA | 1000 nM | NA | NA |
| (760 RU) | | | | | | | |

TABLE E9

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-2H10-A7 | Hu.DLL1 | NA | NA | NA | 1000 nM | NA | NA |
| (522 RU) | | | | | | | |

### Full Kinetics

TABLE E10

| | | | | | Conc. of | | |
|---|---|---|---|---|---|---|---|
| | | | | Rmax | Analyte | | |
| Ligand | Analyte | ka(1/Ms) | kd(1/s) | (RU) | (nM) | KD(M) | Chi² |
| BMH-8D2-D10 | HuDLL4 | 7.59 × 10⁵ | 1.61 × 10⁻³ | 64.5 | 0 | 2.12 × 10⁻⁹ | 0.728 |
| (140 RU) | | | | | 0.78 | | |
| | | | | | 1.56 | | |
| | | | | | 3.125 | | |
| | | | | | 6.25 | | |
| | | | | | 12.5 | | |
| | | | | | 12.5 | | |

TABLE E11

| | | | | | Conc. Of | | |
|---|---|---|---|---|---|---|---|
| | | | | Rmax | Analyte | | |
| Ligand | Analyte | ka(1/Ms) | kd(1/s) | (RU) | (nM) | KD(M) | Chi² |
| BMH-8C2-D4 | HuDLL4 | 1.28 × 10⁶ | 4.39 × 10⁻⁴ | 40.9 | 0 | 3.43 × 10⁻¹⁰ | 0.11 |
| (90 RU) | | | | | 1.56 | | |
| | | | | | 3.125 | | |
| | | | | | 6.25 | | |
| | | | | | 12.5 | | |
| | | | | | 25 | | |

TABLE E12

| | | | | Rmax | Conc. Of | | |
|---|---|---|---|---|---|---|---|
| Ligand | Analyte | ka(1/Ms) | kd(1/s) | (RU) | Analyte | KD(M) | Chi² |
| BMH-2H10-A7 | Hu.DLL4 | NA | NA | NA | 0-100 nM | NA | NA |
| (230 RU) | | | | | | | |

### Conclusions

Based on the full kinetics, it may be concluded that, among the three antibodies tested, 8D2 and 8C2 were observed to bind very well to human DLL4 In contrast, they failed to bind or weakly bound to human DLL1. Among the two antibodies, 8C2 has about 10-fold higher affinity and also an off rate that is 4 fold lower than 8D2.

### C. Results Phase II

### Scouting

TABLE E13

| | | | | Rmax | Conc. Of | | | |
|---|---|---|---|---|---|---|---|---|
| Ligand | Analyte | ka(1/Ms) | kd(1/s) | (RU) | Analyte | | KD(M) | Chi² |
| BMH-8D2-D10 | Porcine.DLL4 | 1.72 × 10⁵ | 1.71 × 10⁻³ | 58.7 | 20 | nM | 9.95 × 10⁻⁹ | 0.713 |
| (80 RU) | | | | | 100 | nM | | |

TABLE E14

| | | | | Rmax | Conc. of | | | |
|---|---|---|---|---|---|---|---|---|
| Ligand | Analyte | ka(1/Ms) | kd(1/s) | (RU) | Analyte | | KD(M) | Chi² |
| BMH-8C2-D10 | Porcine.DLL4 | 1.06 × 10⁵ | 1.62 × 10⁻³ | 63.1 | 20 | nM | 1.54 × 10⁻⁸ | 0.0682 |
| (80 RU) | | | | | 100 | nM | | |

TABLE E15

| | | | | Rmax | Conc. Of | | | |
|---|---|---|---|---|---|---|---|---|
| Ligand | Analyte | ka(1/Ms) | kd(1/s) | (RU) | Analyte | | KD(M) | Chi² |
| BMH-2H10-A7 | Porcine.DLL4 | 2.79 × 10³ | 3.45 × 10⁻³ | 432 | 20 | nM | 1.24 × 10⁻⁶ | 0.0937 |
| (75 RU) | | | | | 100 | nM | | |

### Full Kinetics

TABLE E16

| | | | | | Conc. Of | | |
|---|---|---|---|---|---|---|---|
| | | | | Rmax | Analyte | | |
| Ligand | Analyte | ka(1/Ms) | kd(1/s) | (RU) | (nM) | KD(M) | Chi² |
| BMH-8D2-D10 | Porcine.DLL4 | 2.53 × l0⁵ | 1.82 × 10⁻³ | 68.1 | 0 | 7.21 × 10⁻⁹ | 1.22 |
| (110 RU) | | | | | 3.125 | | |
| | | | | | 12.5 | | |
| | | | | | 25 | | |
| | | | | | 50 | | |
| | | | | | 50 | | |

TABLE E17

| | | | | | Conc. Of | | |
|---|---|---|---|---|---|---|---|
| | | | | Rmax | Analyte | | |
| Ligand | Analyte | ka(1/Ms) | kd(1/s) | (RU) | (nM) | KD(M) | Chi² |
| BMH-8C2-D10 | Porcine.DLL4 | 2.81 × 10⁵ | 1.98 × 10⁻³ | 56.7 | 0 | 7.07 × 10⁻⁹ | 0.652 |
| (80 RU) | | | | | 3.125 | | |
| | | | | | 12.5 | | |
| | | | | | 25 | | |
| | | | | | 50 | | |
| | | | | | 50 | | |

TABLE E18

| | | | | | Conc. of | | |
|---|---|---|---|---|---|---|---|
| | | | | Rmax | Analyte | | |
| Ligand | Analyte | ka(1/Ms) | kd(1/s) | (RU) | nM | KD(M) | Chi² |
| BMH-2H10-A7 | Porcine.DLL4 | 295 | 2.76 × 10⁻³ | 3570 | 0 | 9.37 × 10⁻⁶ | 0.369 |
| (130 RU) | | | | | 25 | | |
| | | | | | 50 | | |
| | | | | | 50 | | |
| | | | | | 100 | | |
| | | | | | 200 | | |

### Example 2: Epitope Binding, 8D2 and 8C2 to DLL4

### A. Materials and Methods

### Materials

Equipment: Biacore 3000
Assay Buffer—10 mM HEPES buffer (pH 7.4), 150 mM NaCl, 3 mM EDTA, 0.05% P20 (polyoxyethylenesorbitan)
Regeneration Buffer—10 mM Glycine buffer (pH 1.75)
Conjugation Buffer—10 mM sodium acetate buffer (pH 5)
Flow rate—The flow rate used for capturing the ligand is 5 μl/min. The flow rate for kinetics analysis is 30 μl/min.

### Abbreviations

| | |
|---|---|
| CM5 chip | Carboxymethylated dextran coated chip |
| SPR | Surface Plasmon Resonance |
| RU | Response Units |
| KD | Equilibrium binding affinity constant. |
| | KD is also defined as the analyte |
| | concentration at which 50% of the |
| | maximum available ligand is in complex |
| | form at equilibrium |
| Analyte | One of the interacting molecules flown |
| | over the surface |
| Ligand | One of the interacting molecules immobilized or |
| | captured on the surface |
| Rmax | Maximum binding capacity (in RU) of ligand |
| | captured/immobilized on the surface |
| MW | Molecular weight |
| HBS-EP | HEPES buffered saline supplemented with |
| | EDTA and P20 |
| ka | Association rate constant in M⁻¹s⁻¹ |
| kd | Dissociation rate constant in s⁻¹ |
| χ² | Chi squared |
| EDC | N-ethyl-N′- (3-dimethyl aminopropyl |
| | carbodiimide) |
| NHS | N-hydroxy succinamide |

### Procedures

Binding experiments were performed on Biacore 3000 at 25° C. Flow cell 2 and 3 of the CM5 chip were coated with human and porcine DLL4 protein using the EDC/NHS amine coupling method as per GE manufacturer's instructions. The unoccupied sites were blocked with 1M ethanolamine. For epitope binding, 1st rat Ab (8D2) was injected and the binding was visible, then an additional 2 injections were made to completely saturate the binding sites. The second Ab (8C2) was then injected. For the second run, he format was reversed by injecting 8C2 first as above to saturation and then injecting 8D2 as indicated to confirm pairing.

### B. Results

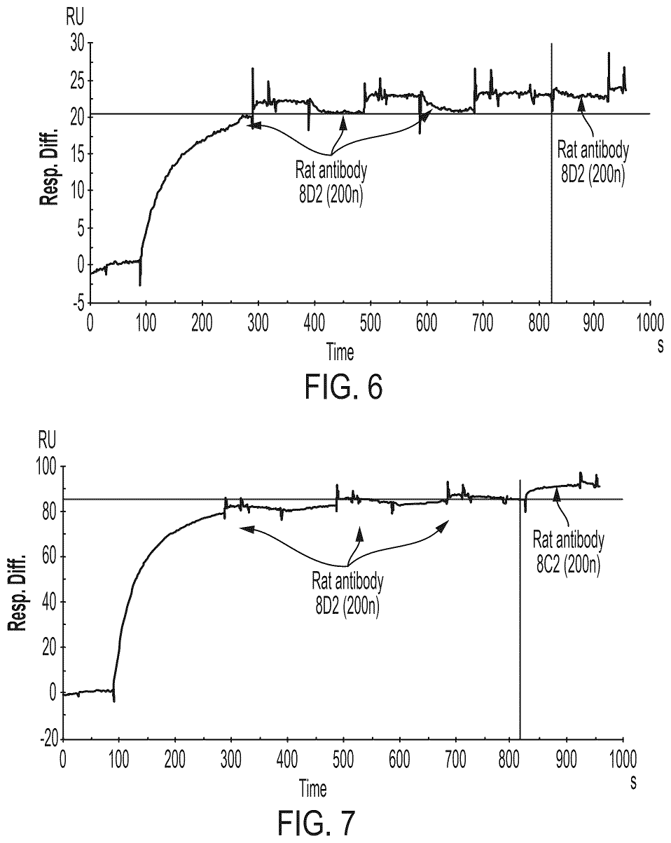

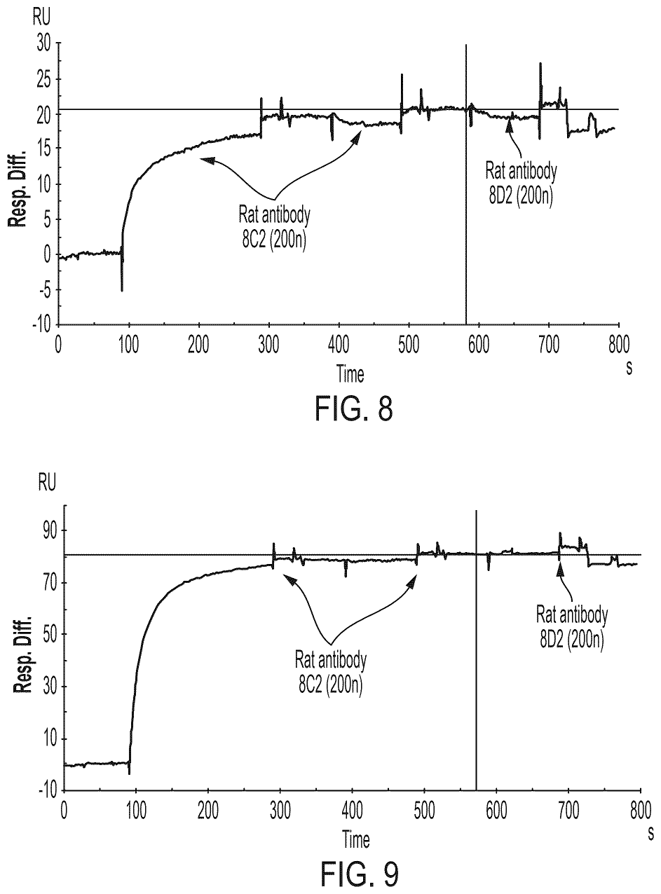

Results in which rat 8D2 was bound first to human DLL4, followed by injection of 8C2 are shown in FIG. 6 and results from experiments in which rat 8C2 was bound to human DDL4 first, followed by injection of 8D2 are shown in FIG. 8. Results in which rat 8D2 was bound first to porcine DLL4, followed by injection of rat 8C2 are shown in FIG. 7 and results from experiments in which rat 8C2 was bound to porcine DDL4 first, followed by injection of rat 8D2 are shown in FIG. 9.

### C. Conclusions

Based on the observations, it may be concluded that the two antibodies, 8D2 and 8C2, may share the same or an overlapping epitope on both porcine and human DLL4. However, some pairing of the Abs with porcine DLL4 was observed only in one format, where 8D2 injected first and then 8C2. The results failed to show the pairing in the reverse format, where 8C2 injected first to saturation and then 8D2.

### Example 3: Sequencing

### A. Experimental Design

### Overview

The procedure used included standard dye-terminator capillary sequencing of cDNA that was generated from extracted mRNA using an RT-PCR protocol. Proprietary primers were used.

### Sequencing Process Overview

Cycle sequencing was performed using BigDye® Terminator v3.1 Cycle Sequencing kits under a standard protocol provided by Life Technologies®. All data was collected using a 3730×l DNA Analyzer system and the Unified Data Collection software provided by Life Technologies® for operation of the 3730×l DNA Analyzer and to collect data produced by the 3730×l DNA Analyzer.

### Contig Assembly and Consensus Reporting

Sequence assembly was performed using CodonCode Aligner (CodonCode Corporation). Mixed base calls are resolved by automatically assigning the most prevalent base call to the mixed base calls. Prevalence is determined by both frequency of a base call and the individual quality of the base calls. Observations are reported in the comments section below.

### Comments

The heavy chain sequence of antibody 10C5 was not sequenced. 5′ RACE resulted in two sequences with similarity to mouse transposing elements.

Due to mis-priming, a shorter amplicon interfered with the sequencing of the heavy chain of antibody 2H10. The desired amplicon was gel extracted and the resulting sequence data was used to resolve mixed base calls.

### B. Results

Sequences of the identified antibody variable regions are shown in the attached Sequence Listing.

All references cited herein are fully incorporated by reference. Having now fully described the invention, it will be understood by those of skill in the art that the invention may be practiced within a wide and equivalent range of conditions, parameters and the like, without affecting the spirit or scope of the invention or any embodiment thereof.

What is claimed is:

1. An isolated IgG antibody that binds to human DLL4 with an affinity at least 1000 times higher than human DLL1, and which comprises:
a) a heavy chain variable region at least 90% identical to the sequence of amino acids 1-114 of SEQ ID NO:14 and/or which includes one or more heavy chain complementarity determining regions (HCDRs) selected from the group consisting of: HCDR1-8C2, amino acids 31-35 of SEQ ID NO:14; HCDR2-8C2, amino acids 50-65 of SEQ ID NO:14; and HCDR3-8C2, amino acids 96-103 of SEQ ID NO:14;
b) a light chain variable region at least 90% identical to the sequence of amino acids 1-106 of SEQ ID NO:16 and/or which includes one or more light chain complementarity determining regions (LCDRs) selected from the group consisting of: LCDR1-8C2, amino acids 23-34 of SEQ ID NO:16; LCDR2-8C2, amino acids 50-56 of SEQ ID NO:16; and LCDR3-8C2, amino acids 89-97 of SEQ ID NO:16.

2. The isolated antibody of claim 1, wherein said antibody is a monoclonal antibody.

3. The antibody of either claim 1 or claim 2, wherein the heavy chain variable region comprises HCDR1-8C2, HCDR2-8C2, and HCDR3-8C2; and the light chain variable region comprises LCDR1-8C2, LCDR2-8C2, and LCDR3-8C2.

4. The antibody of any one of claims 1-3, or a fragment thereof, comprising a ka for human DLL4 of 1.0×10⁵ M⁻¹s⁻¹ or higher.

5. A nucleic acid encoding the antibody of any one of claims 1-4, wherein the heavy chain variable region is encoded by nucleotides 33-374 of SEQ ID NO:13 and the light chain variable region is encoded by nucleotides 1-318 SEQ ID NO:15.

6. The nucleic acid of claim 5, wherein:
a) HCDR1-8C2 is encoded by nucleotides 123-137 SEQ ID NO:13; HCDR2-8C2 is encoded by nucleotides 180-227 of SEQ ID NO:13 and HCDR3-8C2 is encoded by nucleotides 318-341 of SEQ ID NO:13; and
b) LCDR1-8C2 is encoded by nucleotides 70-102 of SEQ ID NO:15; LCDR2-8C2 is encoded by nucleotides 148-168 of SEQ ID NO:15 and LCDR3-8C2 encoded by nucleotides 265-291 of SEQ ID NO:15.

7. A method of treating or preventing a disease or condition in a human patient, wherein said disease or condition is caused or exacerbated by the binding of DLL4 to NOTCH receptors, comprising administering, systemically or locally, to said patient a therapeutically effective amount of the antibody or antibody fragment of any one of claims 1-4.

8. The method of claim 7, wherein said disease or condition is selected from the group consisting of: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

9. The method of claim 7 or 8, wherein said patient is administered a humanized or fully human monoclonal antibody.

10. The method of any one of claims 7-9 wherein said antibody or antibody fragment is administered as a pharmaceutical composition comprising said antibody and a pharmaceutically acceptable carrier.

11. An isolated IgG antibody that binds to human DLL4 with an affinity at least 1000 times higher than human DLL1, and which comprises:
a) a heavy chain variable region at least 90% identical to the sequence of amino acids 1-115 of SEQ ID NO:18 and/or which includes one or more HCDRs selected from the group consisting of: HCDR1-8D2, amino acids 25-29 of SEQ ID NO:18; HCDR2-8D2, amino acids 44-60 of SEQ ID NO: 18; and HCDR3-8D2, amino acids 93-104 of SEQ ID NO:18;
b) a light chain variable region at least 90% identical to the sequence of amino acids 1-110 of SEQ ID NO:20 and/or which includes one or more LCDRs selected from the group consisting of: LCDR1-8D2, amino acids 23-37 of SEQ ID NO:20; LCDR2-8D2, amino acids 53-59 of SEQ ID NO:20; and LCDR3-8D2, amino acids 92-100 of SEQ ID NO:20.

12. The isolated antibody of claim 11, wherein said antibody is a monoclonal antibody.

13. The antibody of claim 11 or 12, wherein the heavy chain variable region comprises HCDR1-8D2, HCDR2-8D2, and HCDR3-8D2; and the light chain variable region comprises LCDR1-8D2, LCDR2-8D2, and LCDR3-8D2.

14. The antibody of any one of claims 11-13 or a fragment thereof, comprising a ka for human DLL4 of 1.0×10⁵ M⁻¹s⁻¹or higher.

15. A nucleic acid encoding the antibody of any one of claims 11-14, wherein the heavy chain variable region is encoded by nucleotides 1-345 of SEQ ID NO:17 and the light chain variable region is encoded by nucleotides 36-365 SEQ ID NO:19.

16. The nucleic acid of claim 15, wherein:
a) HCDR1-8D2 is encoded by nucleotides 73-87 SEQ ID NO:17; HCDR2-8D2 is encoded by nucleotides 130-180 of SEQ ID NO:17 and HCDR3-8D2 is encoded by nucleotides 277-312 of SEQ ID NO:17; and
b) LCDR1-8D2 is encoded by nucleotides 102-146 of SEQ ID NO:19; LCDR2-8D2 is encoded by nucleotides 192-212 of SEQ ID NO:19 and LCDR3-8D2 is encoded by nucleotides 309-335 of SEQ ID NO:19.

17. A method of treating or preventing a disease or condition in a human patient, wherein said disease or condition is caused or exacerbated by the binding of DLL4 to NOTCH receptors, comprising administering, systemically or locally, to said patient a therapeutically effective amount of the antibody or antibody fragment of any one of claims 11-14.

18. The method of claim 17, wherein said disease or condition is selected from the group consisting of: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

19. The method of claim 17 or 18, wherein said patient is administered a humanized or fully human monoclonal antibody.

20. The method of any one of claims 17-19 wherein said antibody or antibody fragment is administered as a pharmaceutical composition comprising said antibody and a pharmaceutically acceptable carrier.

21. An isolated IgG antibody which binds to human DLL4 with an affinity at least 1000 times higher than human DLL1, and which comprises:
a) a heavy chain variable region at least 90% identical to the sequence of amino acids 1-120 of SEQ ID NO:2 and/or which includes one or more HCDRs selected from the group consisting of: HCDR1-2H10, amino acids 31-35 of SEQ ID NO:2; HCDR2-2H10, amino acids 50-65 of SEQ ID NO:2; and HCDR3-2H10, amino acids 98-111 of SEQ ID NO:2; and
b) a light chain variable region at least 90% identical to the sequence of amino acids 1-103 of SEQ ID NO:4 and/or which includes one or more LCDRs selected from the group consisting of: LCDR1-2H10, amino acids 18-28 of SEQ ID NO:4; LCDR2-2H10, amino acids 44-50 of SEQ ID NO:4; and LCDR3-2H10, amino acids 84-91 of SEQ ID NO:4.

22. The isolated antibody of claim 21, wherein said antibody is a monoclonal antibody.

23. The antibody of either claim 21 or 22, wherein the heavy chain variable region comprises HCDR1-2H10, HCDR2-2H10, and HCDR3-2H10; and the light chain variable region comprises LCDR1-2H10, LCDR2-2H10, and LCDR3-2H10.

24. The antibody of any one of claims 21-22 or a fragment thereof, comprising a ka for human DLL4 of 1.0×10⁴ M⁻¹s⁻¹ or higher.

25. A nucleic acid encoding the monoclonal antibody of any one of claim 21-24, wherein the heavy chain variable region is encoded by nucleotides 31-390 of SEQ ID NO:1 and the light chain variable region is encoded by nucleotides 3-311 of SEQ ID NO:3.

26. The nucleic acid of claim 25, wherein:
a) HCDR1-2H10 is encoded by nucleotides 121-135 of SEQ ID NO:1; HCDR2-2H10 is encoded by nucleotides 178-225 of SEQ ID NO:1; and HCDR3-2H10 is encoded by nucleotides 322-363 of SEQ ID NO:1; and
b) LCDR1-2H10 is encoded by nucleotides 54-86 of SEQ ID NO:3; LCDR2-2H10 is encoded by nucleotides 132-152 of SEQ ID NO:3 and LCDR3-2H10 is encoded by nucleotides 249-275 of SEQ ID NO:3.

27. A method of treating or preventing a disease or condition in a human patient, wherein said disease or condition is caused or exacerbated by the binding of DLL4 to NOTCH receptors, comprising administering, systemically or locally, to said patient a therapeutically effective amount of the antibody or antibody fragment of any one of claims 21-24.

28. The method of claim 27, wherein said disease or condition is selected from the group consisting of: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

29. The method of claim 27 or 28, wherein said patient is administered a humanized or fully human monoclonal antibody.

30. The method of any one of claims 27-29 wherein said antibody or antibody fragment is administered as a pharmaceutical composition comprising said antibody and a pharmaceutically acceptable carrier.

31. An isolated IgG antibody which binds to human DLL4 and which comprises:
a) a heavy chain variable region at least 90% identical to the sequence of amino acids 1-120 of SEQ ID NO:6 and/or which includes one or more HCDRs selected from the group consisting of HCDR1-5D7, amino acids 31-35 of SEQ ID NO:6; HCDR2-5D7, amino acids 50-65 of SEQ ID NO:6; and HCDR3-5D7, amino acids 98-109 of SEQ ID NO:6;
b) a light chain variable region at least 90% identical to the sequence of amino acids 1-110 of SEQ ID NO:8 and/or which includes one or more LCDRs selected from the group consisting of: LCDR1-5D7, amino acids 23-37 of SEQ ID NO:8; LCDR2-5D7, amino acids 53-59 of SEQ ID NO:8; and LCDR3-5D7, amino acids 92-100 of SEQ ID NO:8.

32. The isolated antibody of claim 31, wherein said antibody is a monoclonal antibody.

33. The antibody of either claim 31 or claim 32, wherein the heavy chain variable region comprises HCDR1-5D7, HCDR2-5D7, and HCDR3-5D7; and the light chain variable region comprises LCDR1-5D7, LCDR2-5D7, and LCDR3-5D7.

34. The monoclonal antibody of any one of claims 21-33, or a fragment thereof, comprising a ka for human DLL4 of 1.0×10⁴ M⁻¹s⁻¹ or higher.

35. A nucleic acid encoding the monoclonal antibody of any one of claim 31-34, wherein the heavy chain variable region is encoded by nucleotides 55-414 of SEQ ID NO:5 and the light chain variable region is encoded by nucleotides 23-352 SEQ ID NO:7.

36. The nucleic acid of claim 35, wherein:
a) HCDR1-5D7 is encoded by nucleotides 145-159 SEQ ID NO:5; HCDR2-5D7 is encoded by nucleotides 202-249 of SEQ ID NO:5 and HCDR3-5D7 is encoded by nucleotides 346-381 of SEQ ID NO:5; and
b) LCDR1-5D7 is encoded by nucleotides 89-133 of SEQ ID NO:7; LCDR2-5D7 is encoded by nucleotides 179-199 of SEQ ID NO:7 and LCDR3-5D7 is encoded by nucleotides 296-322 of SEQ ID NO:7.

37. A method of treating or preventing a disease or condition in a human patient, wherein said disease or condition is caused or exacerbated by the binding of DLL4 to NOTCH receptors, comprising administering, systemically or locally, to said patient a therapeutically effective amount of the antibody or antibody fragment of any one of claims 31-34.

38. The method of claim 37, wherein said disease or condition is selected from the group consisting of: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

39. The method of claim 37 or 38, wherein said patient is administered a humanized or fully human monoclonal antibody.

40. The method of any one of claims 37-39 wherein said antibody of antibody fragment is administered as a pharmaceutical composition comprising said antibody and a pharmaceutically acceptable carrier.

41. An isolated IgG antibody which binds to human DLL4 and which comprises:
a) a heavy chain variable region at least 90% identical to the sequence of amino acids 1-114 of SEQ ID NO:10 and/or which includes one or more HCDRs selected from the group consisting of HCDR1-8B2, amino acids 20-29 of SEQ ID NO:10; HCDR2-8B2, amino acids 44-53 of SEQ ID NO:10; and HCDR3-8B2, amino acids 93-103 of SEQ ID NO:10;
b) a light chain variable region at least 90% identical to the sequence of amino acids 1-107 of SEQ ID NO:12 and/or which includes one or more LCDRs selected from the group consisting of LCDR1-8B2, amino acids 24-34 of SEQ ID NO:12; LCDR2-8B2, amino acids 50-56 of SEQ ID NO:12; and LCDR3-8B2, amino acids 89-97 of SEQ ID NO:12.

42. The isolated antibody of claim 41, wherein said antibody is a monoclonal antibody.

43. The antibody of either claim 41 or claim 42, wherein the heavy chain variable region comprises HCDR1-8B2, HCDR2-8B2, and HCDR3-8B2; and the light chain variable region comprises LCDR1-8B2, LCDR2-8B2, and LCDR3-8B2.

44. The antibody of any one of claims 41-43 or fragment thereof, comprising a ka for human DLL4 of 1.0×10⁴ M⁻¹s⁻¹ or higher.

45. A nucleic acid encoding the monoclonal antibody of any one of claims 41-44, wherein the heavy chain variable region is encoded by nucleotides 1-342 of SEQ ID NO:9 and the light chain variable region is encoded by nucleotides 141-461 SEQ ID NO:11.

46. The nucleic acid of claim 45, wherein:
a) HCDR1-8B2 is encoded by nucleotides 58-87 SEQ ID NO:9; HCDR2-8B2 is encoded by nucleotides 130-159 of SEQ ID NO:9 and HCDR3-8B2 is encoded by nucleotides 277-309 of SEQ ID NO:9; and
b) LCDR1-8B2 is encoded by nucleotides 210-242 of SEQ ID NO:11; LCDR2-8B2 is encoded by nucleotides 288-308 of SEQ ID NO:11 and LCDR3-8B2 is encoded by nucleotides 405-431 of SEQ ID NO:11.

47. A method of treating or preventing a disease or condition in a human patient, wherein said disease or condition is caused or exacerbated by the binding of DLL4 to NOTCH receptors, comprising administering, systemically or locally, to said patient a therapeutically effective amount of the antibody or antibody fragment of any one of claims 41-44.

48. The method of claim 47, wherein said disease or condition is selected from the group consisting of: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

49. The method of claim 47 or 48, wherein said patient is administered a humanized or fully human monoclonal antibody.

50. The method of any one of claims 47-49 wherein said antibody of antibody fragment is administered as a pharmaceutical composition comprising said antibody and a pharmaceutically acceptable carrier.

51. An isolated IgG antibody which binds to human DLL4 and which comprises:
a light chain variable region at least 90% identical to the sequence of amino acids 1-102 of SEQ ID NO:22 and/or which includes one or more LCDRs selected from the group consisting of LCDR1-10C5, amino acids 19-29 of SEQ ID NO:22; LCDR2-10C5, amino acids 45-51 of SEQ ID NO:22; and LCDR3-10C5, amino acids 84-92 of SEQ ID NO:22.

52. The isolated antibody of claim 51, wherein said antibody is a monoclonal antibody.

53. The monoclonal antibody of either claim 51 or 52, wherein the light chain variable region comprises LCDR1-10C5, LCDR2-10C5, and LCDR3-10C5.

54. The monoclonal antibody of any one of claims 51-53 or a fragment thereof, comprising a ka for human DLL4 of 1.0×10⁴ M⁻¹s⁻¹ or higher.

55. A nucleic acid encoding the monoclonal antibody of any one of claim 51-54, wherein the light chain variable region is encoded by nucleotides is encoded by nucleotides 1-306 SEQ ID NO:21.

56. The nucleic acid of claim 55, wherein:
LCDR1-10C5 is encoded by nucleotides 55-87 of SEQ ID NO:21; LCDR2-10C5 is encoded by nucleotides 133-153 of SEQ ID NO:21 and LCDR3-10C5 is encoded by nucleotides 250-276 of SEQ ID NO:21.

57. A method of treating or preventing a disease or condition in a human patient, wherein said disease or condition is caused or exacerbated by the binding of DLL4 to NOTCH receptors, comprising administering, systemically or locally, to said patient a therapeutically effective amount of the antibody or antibody fragment of any one of claims 46-49.

58. The method of claim 57, wherein said disease or condition is selected from the group consisting of: cardiovascular disease, including atherogenesis, atherosclerosis and vascular calcification; vein graft disease, arteriovenous fistula failure; nonalcoholic fatty liver disease; obesity; metabolic disease; type 1 and type 2 diabetes; cancer, including colorectal, breast and lung cancer; tumor cell metastasis; arthritis; autoimmune diseases such as rheumatoid arthritis, scleroderma, and systemic lupus erythematosus; failure or dysfunction of artificial or tissue-engineered tissues or organs; and rejection or graft-versus-host disease after organ or cell transplantation.

59. The method of claim 57 or 58, wherein said patient is administered a humanized or fully human monoclonal antibody.

60. The method of any one of claims 57-59 wherein said antibody of antibody fragment is administered as a pharmaceutical composition comprising said antibody and a pharmaceutically acceptable carrier.

61. The antibody or fragment of any one of claim 1-4, 11-14, 21-24, 31-34, 41-44 or 51-54, wherein said antibody or fragment comprises a post-translation modification.

62. The antibody or fragment of claim 61, wherein said post-translation modification is selected from the group consisting of: PEGylation; phosphorylation; methylation; acetylation; ubiquitination; nitrosylation; glycosylation; and lipidation.

63. The antibody or fragment of any one of claim 1-4, 11-14, 21-24, 31-34, 41-44 or 51-54, wherein said antibody or fragment further comprises a detectable label.

64. The antibody or fragment of claim 57, wherein said detectable label is selected from the group consisting of: a radioactive label; a fluorophore; a chemiluminescent label; an enzymatic label; biotin; avidin; and a heavy metal.

65. The antibody or fragment of claim 64, wherein said detectable label is an enzyme selected from the group consisting of: alkaline phosphatase and horseradish peroxidase.

66. An immunoassay comprising the step of binding the antibody or fragment of any one of claim 1-4, 11-14, 21-24, 31-34, 41-44 or 51-54 to DLL4.

67. The immunoassay of claim 66, wherein DLL4 is human DLL4 and the antibody or fragment further comprises a detectable label.

68. The immunoassay of claim 66, wherein DLL4 is human DLL4 and the immunoassay further comprises a step of detecting the antibody or fragment by binding a detectably labeled second antibody to the antibody or fragment after it has bound to said human DLL4.

69. The immunoassay of any one of claims 66-68, selected from the group consisting of: an immunohistochemistry blot; a Western blot; fluorescence-activated cell sorting; a chemiluminescence immunoassay; a radioimmunoassay; an enzyme linked immunosorbent assay; an immuno-precipitation assay; and immuno-electron microscopy.

70. A method of detecting or quantitating DLL4-expressing cells in a test sample from a subject, comprising assaying said test sample for cells expressing DLL4 using an immunoassay in which the antibody or antibody fragment of any one of claim 1-4, 11-14, 21-24, 31-34, 41-44 or 51-54 binds to said DLL4.

71. The method of claim 70, wherein said test sample is a biopsy sample, tissue sample, or a bodily fluid.

72. The method of claim 70 or 71, wherein said subject has a disease or condition selected from the group consisting of: cardiovascular disease; vein graft disease; arteriovenous fistula failure; nonalcoholic fatty liver disease; obesity; metabolic disease; type 1 or type 2 diabetes; cancer, and transplanted tissues or organs.

73. A method of studying the effect of DLL4 on a normal or pathological process, comprising administering the antibody or antibody fragment of any one of claim 1-4, 11-14, 21-24, 31-34, 41-44 or 51-54 to test animals used as a model of said normal or pathological process and comparing the effect of such administration to control animals that do not receive the antibody or antibody fragment.


SEQUENCE LISTING (22 sequences)

SEQ ID NO: 1 - DNA, 483 bp, Rattus norvegicus
  misc_feature 31..120: note = Codes for heavy chain framework region 1 (HFR1-2H10)
  misc_feature 121..135: note = Codes for complementarity determining region 1 (HCDR1-2H10)
  misc_feature 136..177: note = Codes for HFR2-2H10
  misc_feature 178..225: note = Codes for HCDR2-2H10
  misc_feature 226..321: note = Codes for HFR3-2H10
  misc_feature 322..363: note = Codes for HCDR3-2H10
  misc_feature 364..390: note = Codes for HFR4-2H10
  source 1..483: mol_type = genomic DNA
  CDS 31..390: note = Heavy chain variable region of monoclonal antibody 2H10
ctggtgacatttccaagctgtgtcctgtcccaggtgcaggtgaaagagtcaggacctggtctggtgcagccctcacagaccctgtctctcacctgcattgtctctgggttctcactaagcagctatcatgtaagctgggttcgccagcctcctggaaagagtctggtgtggatgggaacaatatgggctggtggaggtacaaattataattcggctgtacaatcccgactgagcatcagccgggacacctccaagagccaagttttcttaaaagtgaacagtctgcaacctgaagacacaggcacttactactgtgccagacattactatgatggttattatcacggcccctactttgattactggggccaaggagtcatggtcacagtctcctcagcccaaacaacagccccatctgtctatccactggctcctggatgtggtgatacaaccagctccacggtgactctgggatgcctggtc

SEQ ID NO: 2 - protein, 120 aa, Rattus norvegicus
QVQVKESGPGLVQPSQTLSLTCIVSGFSLSSYHVSWVRQPPGKSLVWMGTIWAGGGTNYNSAVQSRLSISRDTSKSQVFLKVNSLQPEDTGTYYCARHYYDGYYHGPYFDYWGQGVMVTV

SEQ ID NO: 3 - DNA, 338 bp, Rattus norvegicus
  misc_feature 3..53: note = Codes for light chain framework region 1 (LFR1-2H10)
  misc_feature 54..86: note = Codes for light chain complementarity determining region 1 (LCDR1-2H10)
  misc_feature 87..131: note = Codes for LFR2-2H10
  misc_feature 132..152: note = Codes for LCDR2-2H10
  misc_feature 153..248: note = Codes for LFR3-2H10
  misc_feature 249..275: note = Codes for LCDR3-2H10
  misc_feature 276..311: note = Codes for LFR4-2H10
  source 1..338: mol_type = genomic DNA
  CDS 3..311: note = Light chain variable region of monoclonal antibody 2H10
agtctccagcttccctgtctgcatctctgggagaaactgtcaccttcgaatgtcgagcaagtgaggacatttacactaatttagcgtggtatcagcagaaaccagggaactctcctcagctcctgatctatgatgcaaataccttggcagatggggtcccatcacggttcagtggcagtggatctggcacacagttttctctaaagattaacagcctgcaatctgaagatgtcgccagttatttctgtcaacaatatgacagttatccgtggacgttcggtggaggcaccaagctggaattgagacgggctgatgctgcaccaactgtatccatctcc

SEQ ID NO: 4 - protein, 103 aa, Rattus norvegicus
SPASLSASLGETVTFECRASEDIYTNLAWYQQKPGNSPQLLIYDANTLADGVPSRFSGSGSGTQFSLKINSLQSEDVASYFCQQYDSYPWTFGGGTKLELRRA

SEQ ID NO: 5 - DNA, 485 bp, Rattus norvegicus
  misc_feature 55..144: note = Codes for heavy chain framework region 1 (HFR1-5D7)
  misc_feature 145..159: note = Codes for heavy chain complementarity determining region 1 (HCDR1-5D7)
  misc_feature 160..201: note = Codes for HFR2-5D7
  misc_feature 202..249: note = Codes for HCDR2-5D7
  misc_feature 250..345: note = Codes for HFR3-5D7
  misc_feature 346..381: note = Codes for HCDR3-5D7
  misc_feature 382..414: note = Codes for HFR4-5D7
  source 1..485: mol_type = genomic DNA
  CDS 55..414: note = Heavy chain variable region for monoclonal antibody 5D7
aaattcctggtgctgttgctctgcctggtgacatttccaagctgtgtcctgtcccaggtacagctgaaggagtcaggacctggcctggtgcagccctcagagaccctgtccctcacctgcactgtctctgggttctcactaaccaggtatgctgtaaactgggttcgacagcctccaggaaaaggtctggagtggatgggaagagtgatggatgatggagacacatcacacaattcagctctcaattcccgactgaacatcagcagggacacctccaagaaccaagttttcttaaagatgaacagtctgcaaactgaagacccaggcacttatttctgtgccagagagggagatggtttttcatggtatggtatggatgcctggggtcaaggaacttcagtcactgtctcctcagcccaaacaacagccccatctgtctatccactggctcctggatgtggtgatacaaccagctccacggtgac

SEQ ID NO: 6 - protein, 120 aa, Rattus norvegicus
QVQLKESGPGLVQPSETLSLTCTVSGFSLTRYAVNWVRQPPGKGLEWMGRVMDDGDTSHNSALNSRLNISRDTSKNQVFLKMNSLQTEDPGTYFCAREGDGFSWYGMDAWGQGTSVTVSS

SEQ ID NO: 7 - DNA, 391 bp, Rattus norvegicus
  misc_feature 23..88: note = Codes for light chain framework region 1 (LFR1-5D7)
  misc_feature 89..133: note = Codes for complementarity dtermining region 1 (LCDR1-5D7)
  misc_feature 134..178: note = Codes for LFR2-5D7
  misc_feature 179..199: note = Codes for LCDR2-5D7
  misc_feature 200..295: note = Codes for LFR3-5D7
  misc_feature 296..322: note = Codes for HCDR3-5D7
  misc_feature 323..352: note = Codes for LFR4-5D7
  source 1..391: mol_type = genomic DNA
  CDS 23..352: note = Light chain variable region for monoclonal antibody 5D7
gtgggttccagcctccactggtgacattgtgctgacccagtctcctgctttggctgtgtctctagggcagagggccacaatctcctgtagagccagccaaagtgtcagtatatctagctataatctcatgcagtggtaccaacagaaaccaggacaccagcccaaactcctcgtctatgctgcatccaaccttgcatctgggatccctgccaggttcagtggcagtgggtctgggacagacttcgccctcaccattgatcctgtgcaggctgatgatgttgcaacctatcactgtcagcagagtaaggatcatcctccgacgttcggtggaggcaccaagctggaattgaaacgggctgatgctgcaccaactgtatccatctcccaccat

SEQ ID NO: 8 - protein, 110 aa, Rattus norvegicus
DIVLTQSPALAVSLGQRATISCRASQSVSISSYNLMQWYQQKPGHQPKLLVYAASNLASGIPARFSGSGSGTDFALTIDPVQADDVATYHCQQSKDHPPTFGGGTKLELK

SEQ ID NO: 9 - DNA, 436 bp, Rattus norvegicus
  misc_feature 1..57: note = Codes for heavy chain framework region 1 (HFR1-8B2)
  misc_feature 58..87: note = Codes for heavy chain complementarity determining region 1 (HCDR1-8B2)
  misc_feature 88..129: note = Codes for HFR2-8B2
  misc_feature 130..159: note = Codes for HCDR2-8B2
  misc_feature 160..276: note = Codes for HFR3-8B2
  misc_feature 277..309: note = Codes for HCDR3-8B2
  misc_feature 310..342: note = Codes for HFR4-8B2
  source 1..436: mol_type = genomic DNA
  CDS 1..342: note = Heavy chain variable region for monoclonal antibody 8B2
tctggagctgagctggtgaagcctgggacttctgtgaagctgtcctgcaggacttctggctacacctttactaacaaccatatgaactggataaagcagacgactggacagggccttgagtgggttggaattattaatccgggaagtggacgtactcactacaatgtgaagttcaagggcaaggccacattgactgtagacaaatcctccagcacagccttcatgcaactcagcagcctgacacctgaggactctgcggtctattactgtgcaagaaggggtgacaactctgactacgctatggatacctggggtcaaggaacttcagtcactgtctcctcagcccaaacaacagccccatctgtctatccactggctcctggatgtggtgatacaaccagctccacggtgactctgggatgcctggtcaagggct

SEQ ID NO: 10 - protein, 114 aa, Rattus norvegicus
SGAELVKPGTSVKLSCRTSGYTFTNNHMNWIKQTTGQGLEWVGIINPGSGRTHYNVKFKGKATLTVDKSSSTAFMQLSSLTPEDSAVYYCARRGDNSDYAMDTWGQGTSVTVSS

SEQ ID NO: 11 - DNA, 488 bp, Rattus norvegicus
  misc_feature 141..209: note = Codes for light chain framework region 1 (LFR1-8B2)
  misc_feature 210..242: note = Codes for light chain complementarity determining region 1 (LCDR1-8B2)
  misc_feature 243..287: note = Codes for LFR2-8B2
  misc_feature 288..308: note = Codes for LCDR2-8B2
  misc_feature 309..404: note = Codes for LFR3-8B2
  misc_feature 405..431: note = Codes for LCDR3-8B2
  misc_feature 432..461: note = Codes for LFR4-8B2
  source 1..488: mol_type = genomic DNA
  CDS 141..461: note = Light chain variable region of monoclonal antibody 8B2
taatacgactcactatagggcaagcagtggtatcaacgcagagtacgggggaaatgcatcagaccagcatgggcatcaagatggaatcacagactctggtcttcatatccatactgctctggttatatggagctgatgggaacattgtaatgacccaatctcccaaatccatgtccatgtcagtaggagagagggtcaccttgacctgcaaggccagtgagaatgtggttacttatgtttcctggtatcaacagaaaccagagcagtctcctaaactgctgatatacggggcatccaaccggtacactggggtccccgatcgcttcacaggcagtggatctgcaacagatttcactctgaccatcagcagtgtgcaggctgaagaccttgcagattatcactgtggacagggttacagctatccgtacacgttcggaggggggaccaagctggaaataaaacgggctgatgctgcaccaactgtatcc

SEQ ID NO: 12 - protein, 107 aa, Rattus norvegicus
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIK

SEQ ID NO: 13 - DNA, 445 bp, Rattus norvegicus
  misc_feature 33..122: note = Codes for heavy chain framework region 1 (HFR1-8C2)
  misc_feature 123..137: note = Codes for heavy chain complementarity determining region 1 (HCDR1-8C2)
  misc_feature 138..179: note = Codes for HFR2-8C2
  misc_feature 180..227: note = Codes for HCDR2-8C2
  misc_feature 228..317: note = Codes for HFR3-8C2
  misc_feature 318..341: note = Codes for HCDR3-8C2
  misc_feature 342..374: note = Codes for HFR4-8C2
  source 1..445: mol_type = genomic DNA
  CDS 33..374: note = Codes for heavy chain variable region of monoclonal antibody 8C2
gcctggtgacattcccaagctgtgtcctgtcccaggtgcagctgaaggagtcaggacctggtctggtgcagccctcacagaccctgtccctcacctgcactgtctctggaatctcaataagcatttatggtgttagctgggttcgccagcctccaggaaagggtctggagtggatgggaggaatatggggtgatggatccacagattataattcagctctcaaatcccgactgagcctcagcagggacacctccaagagccaagttttcttaaaaatgaacagtctgcaaactgaagacacagccatttacttctgtaccctcagtggggactttgattactggggccacggagtcttggtcacagtctcctcagcccaaacaacagccccatctgtctatccactggctcctggatgtggtgatacaaccagctccacggtgac

SEQ ID NO: 14 - protein, 114 aa, Rattus norvegicus
QVQLKESGPGLVQPSQTLSLTCTVSGISISIYGVSWVRQPPGKGLEWMGGIWGDGSTDYNSALKSRLSLSRDTSKSQVFLKMNSLQTEDTAIYFCTLSGDFDYWGHGVLVTVSS

SEQ ID NO: 15 - DNA, 349 bp, Rattus norvegicus
  misc_feature 1..69: note = Codes for light chain framework region 1 (LFR1-8C2)
  misc_feature 70..102: note = Codes for light chain complementarity determining region 1 (LCDR1-8C2)
  misc_feature 103..147: note = Codes for LFR2-8C2
  misc_feature 148..168: note = Codes for LCDR2-8C2
  misc_feature 169..264: note = Codes for LFR3-8C2
  misc_feature 265..291: note = Codes for LCDR3-8C2
  misc_feature 292..318: note = Codes for LFR4-8C2
  source 1..349: mol_type = genomic DNA
  CDS 1..318: note = Light chain variable region of monoclonal antibody 8C2
gacatccagttgacccagtctcctgcctccctgtctgcatctctggatgaaattgtcaccatcacatgccaggcaagcctggacattggtaattggttagcatggtatcagcagaaaacagggaaatctcctcaactcctgatctatggtgcaaccagcttggcgggtggggtcccatcaaggttcagcggcagtagatctggcacacagtattctcttaagatcagcaaactacaggttgaagatactggaatctattactgtctacagcattatcgtgctccattcacgttcggctcagggacgaagttggaaataaaacgggctgatgctgcaccaactgtatcca

SEQ ID NO: 16 - protein, 106 aa, Rattus norvegicus
DIQLTQSPASLSASLDEIVTITCQASLDIGNWLAWYQQKTGKSPQLLIYGATSLAGGVPSRFSGSRSGTQYSLKISKLQVEDTGIYYCLQHYRAPFTFGSGTKLEI

SEQ ID NO: 17 - DNA, 438 bp, Rattus norvegicus
  misc_feature 1..72: note = Codes for heavy chain framework region 1 (HFR1-8D2)
  misc_feature 73..87: note = Codes for heavy chain complementarity determining region 1 (HCDR1-8D2)
  misc_feature 88..129: note = Codes for HFR2-8D2
  misc_feature 130..180: note = Codes for HCDR2-8D2
  misc_feature 181..276: note = Codes for HFR3-8D2
  misc_feature 277..312: note = Codes for HCDR3-8D2
  misc_feature 313..345: note = Codes for HFR4-8D2
  source 1..438: mol_type = genomic DNA
  CDS 1..345: note = Heavy chain variable region for monoclonal antibody 8D2
tctgggggaggcttagtgcagcctggaaggtccatgaagctctcctgtgcagcctcaggattcactttcagtaactatgacatggcctgggtccgccaggatccaaagaagggtctggaatgggtcgcaacaatcagttatgatggtagaagcacttactatcgagactccgtgaagggccgattcactatttccagagataatgcaaaaagtaccctatatctgcaaatggacagtctgaggtctgaggacacggccacttattactgtaaggactatgatgggcattatcacccctatggtatggaggcctggggtcaaggaatttcagtcactgtctcctcagcccaaacaacagccccatctgtctatccactggctcctggatgtggtgatacaaccagctccacggtgactctgggatgcctggtcaagggc

SEQ ID NO: 18 - protein, 115 aa, Rattus norvegicus
SGGGLVQPGRSMKLSCAASGFTFSNYDMAWVRQDPKKGLEWVATISYDGRSTYYRDSVKGRFTISRDNAKSTLYLQMDSLRSEDTATYYCKDYDGHYHPYGMEAWGQGISVTVSS

SEQ ID NO: 19 - DNA, 402 bp, Rattus norvegicus
  misc_feature 36..101: note = Codes for light chain framework region 1 (LFR1-8D2)
  misc_feature 102..146: note = Codes for light chain complementarity region 1 (LCDR1-8D2)
  misc_feature 147..191: note = Codes for LFR2-8D2
  misc_feature 192..212: note = Codes for LCDR2-8D2
  misc_feature 213..308: note = Codes for LFR3-8D2
  misc_feature 309..335: note = Codes for LCDR3-8D2
  misc_feature 336..365: note = Codes for LFR4-8D2
  source 1..402: mol_type = genomic DNA
  CDS 36..365: note = Light chain variable region of monoclonal antibody 8D2
ggttgctgctgctgtgggttccaggctccactggtgacattgtgctgacccagtctcctgctttggctgtgactctagggcagagggccacaatctcctgtagagccagccagagtgtcactatatctaactataatctcatgcagtggtaccaacagagaccaggacggcaacccaaactcctcatctatgatgcatccaacctagcatctgggatccctgccaggttcagtggcagtgggtctgggacagacttcaccctcaccattgatcctgtgcaggctgatgatattgcaacctattactgtcagcagagtagggatgatcctcggacgttcggtggaggcaccaagctggaattgagacgggctgatgctgcaccaactgtatccatctcccacc

SEQ ID NO: 20 - protein, 110 aa, Rattus norvegicus
DIVLTQSPALAVTLGQRATISCRASQSVTISNYNLMQWYQQRPGRQPKLLIYDASNLASGIPARFSGSGSGTDFTLTIDPVQADDIATYYCQQSRDDPRTFGGGTKLELR

SEQ ID NO: 21 - DNA, 333 bp, Rattus norvegicus
  misc_feature 1..54: note = Codes for light chain framework region 1 (LFR1-10C5)
  misc_feature 55..87: note = Codes for light chain complementarity determining region 1 (LCDR1-10C5)
  misc_feature 88..132: note = Codes for LFR2-10C5
  misc_feature 133..153: note = Codes for LCDR2-10C5
  misc_feature 154..249: note = Codes for LFR3-10C5
  misc_feature 250..276: note = Codes for LCDR3-10C5
  misc_feature 277..306: note = Codes for LFR4-10C5
  source 1..333: mol_type = genomic DNA
  CDS 1..306: note = Light chain variable region of monoclonal antibody 10C5
cagtctcctgcctccctgtctgcatctctggaagaaattgtcaccatcacatgccaggcaagccaggacattggtaattggttatcatggtatcagcagaaaccagggaaatctcctcagctcctgatctatggtgcaaccagcttggcagatggggtcccatcaaggttcagcggcagtagatctggcacacagtattctcttaagatcagcagactacaggttgaagatattggaatctattactgtctacaggcttatagtgctcctcggacgttcggtggaggcaccaagctggaattgaaacgggctgatgctgcaccaactgtatcc

SEQ ID NO: 22 - protein, 102 aa, Rattus norvegicus
QSPASLSASLEEIVTITCQASQDIGNWLSWYQQKPGKSPQLLIYGATSLADGVPSRFSGSRSGTQYSLKISRLQVEDIGIYYCLQAYSAPRTFGGGTKLELK